(19) United States
(12) Patent Application Publication
(10) Pub. No.: US 2022/0041691 A1
(43) Pub. Date: Feb. 10, 2022

(54) METHODS FOR DIAGNOSING OR TREATING POST-TRAUMATIC STRESS DISORDER, AND COMPOSITIONS THEREFOR

(71) Applicant: Centre for Addiction and Mental Health, Toronto (CA)

(72) Inventors: Fang LIU, Toronto (CA); Haiyin LI, Toronto (CA)

(21) Appl. No.: 17/281,509

(22) PCT Filed: Jul. 17, 2019
(86) PCT No.: PCT/CA2019/050987

§ 371 Date: Mar. 30, 2021

(30) Foreign Application Priority Data
Oct. 5, 2018 (CA) 3019970

(51) Int. Cl. C07K 14/72; A61P 25/00; G01N 33/94; C12N 9/90

(52) U.S. Cl. CPC C07K 14/721; A61P 25/00; G01N 33/9493; C12Y 502/01008; A61K 38/00; G01N 2333/723; G01N 2500/02; G01N 2800/30; C12N 9/90

(57) ABSTRACT

Provided herein are methods for preventing or treating post-traumatic stress disorder (PTSD) in a subject, the methods including: administering an agent which inhibits formation of a glucocorticoid receptor (GR)-FK506 Binding Protein 51 (FKBP51) complex (GR-FKBP51 complex), or which disrupts already formed GR-FKBP51 complex, to the subject; thereby reducing a level of GR-FKBP51 complex in the subject and preventing or treating the PTSD. Also provided are methods for diagnosing a subject as having, or being at risk of developing, a PTSD, the methods including steps of: measuring a level of a GR-FKBP51 complex in the subject; comparing the measured level to a reference level of a non-PTSD condition; and identifying the subject as having, or being at risk of developing, PTSD where the measured level is elevated relative to the reference level. Agents, compositions, and/or kits for the diagnosis and/or treatment of PTSD are also described.

### FIELD OF INVENTION

The present invention relates generally to the diagnosis and treatment of post-traumatic stress disorder (PTSD). More specifically, the present invention relates to methods for diagnosing and/or treating PTSD, and compositions therefor.

### BACKGROUND

Posttraumatic stress disorder (PTSD) was originally described in soldiers exposed to horrific battlefield events, but the conception of psychological trauma has since been expanded to include any life or limb-threatening events in either the military or civilian population. Posttraumatic stress disorder (PTSD) develops after a person is exposed to one or more traumatic events, such as sexual assault, warfare, traffic collisions, terrorism or other threats on a person's life. PTSD symptoms may include flashbacks and nightmares, avoidance of reminders of the trauma, hyperarousal, and insomnia.¹ PTSD affects approximately one fifth of military combat veterans² and victims of physical assault.³ Approximately 2% of US military personnel report having PTSD symptoms.⁴ In Canada, the lifetime prevalence of PTSD in the general population has been estimated to be 9%.⁵

The most effective current treatment for PTSD is cognitive-behavioural psychotherapy aimed at desensitizing the patient to cues related to the traumatic event through gradual and repeated exposure.^{6, 7} Medications are generally less effective than therapy, and only two medications are currently approved by the U.S. Food and Drug Administration (FDA) for the treatment of PTSD: sertraline and paroxetine; both are selective serotonin re-uptake inhibitor antidepressants.⁸ Unfortunately, overall functioning and outcomes for patients with PTSD remain poor,^{9, 10} and stable remission of chronic PTSD is rare.¹¹ PTSD is unusual among psychiatric disorders in having a clear precipitant, followed by a delay in onset of symptoms, and this interval presents a promising opportunity for preventative interventions in a readily identifiable at-risk population, however taking advantage of this interval has so far proven challenging.

The molecular mechanisms underlying PTSD have remained largely unknown, and this has hampered development of treatment approaches, as well as the development of tests which can reliably predict the onset of PTSD. An emerging pathway of potential relevance may involve the FK506 binding protein 5 (FKBP5) gene.¹² FKBP5 is a negative-feedback regulator of the glucocorticoid receptor (GR), so high levels of FKBP5 reduces available glucocorticoid receptors and promotes glucocorticoid resistance. A specific FKBP5 gene variant (rs1360780) affects susceptibility to PTSD after early-life trauma through modifying glucocorticoid binding to this gene.^{13, 14} This in turn leads to demethylation of an intron 7 CpG site in FKBP5, resulting in persistent FKBP5 activation.¹² The glucocorticoid release triggered by traumatic events in adulthood further activates the demethylated form of FKBP5 and leads to glucocorticoid resistance, which is believed to contribute to the symptoms of PTSD through promoting hyperarousal of the stress-response system.¹⁵ To date, effective therapeutic approaches, as well as reliable diagnostic approaches, for PTSD are lacking.

Alternative, additional, and/or improved methods for the treatment and/or diagnosis of PTSD, and/or agents, compositions, and/or kits therefor, are desirable.

### SUMMARY OF INVENTION

As described in detail hereinbelow, it has now been identified that a protein complex can be formed by glucocorticoid receptor (GR) and FK506 Binding Protein 51 (FKBP51; aka FKBP5), and that this complex (herein referred to as GR-FKBP51 complex) is elevated in peripheral blood from PTSD patients as compared to healthy control subjects as well as subjects exposed to trauma but who did not develop PTSD. Furthermore, compounds which may inhibit formation of GR-FKBP51 complex, and/or which may disrupt already formed GR-FKBP51 complex, have now been developed, and are described in detail herein. As described in Example 1 below, testing was performed in a mouse model, where freezing time in fear-conditioned mice (an animal model of PTSD) was significantly reduced when administered immediately after the fear-conditioning training, and after the extinction session. Increased levels of GR-FKBP52 complex, and nuclear translocation of GR, was also observed. These developments may provide for methods of preventing or treating PTSD, methods for diagnosing PTSD, and/or agents, compositions, and/or kits therefor.

In one embodiment, there is provided herein a method for preventing or treating post-traumatic stress disorder (PTSD) in a subject in need thereof, said method comprising:
administering an agent which inhibits formation of a glucocorticoid receptor (GR)-FK506 Binding Protein 51 (FKBP51) complex (GR-FKBP51 complex), or which disrupts already formed GR-FKBP51 complex, to the subject;
thereby reducing a level of GR-FKBP51 complex in the subject and preventing or treating the PTSD.

In another embodiment of the above method, the agent may comprise:
a competitive binder for a region of GR which binds FKBP51 in the GR-FKBP51 complex; or
a competitive binder for a region of FKBP51 which binds GR in the GR-FKBP51 complex.

In yet another embodiment of the above method or methods, the agent may comprise a mimic of an N-terminal region of GR (GR_{NT}; SEQ ID NO: 2) which interacts with FKBP51 in the GR-FKBP51 complex.

In still another embodiment of the above method or methods, the agent may comprise a mimic of a GR_{NT-4} (SEQ ID NO: 3) or GR_{NT-4-1} (SEQ ID NO: 4) region of GR.

In another embodiment of the above method or methods, the agent may comprise a polypeptide having at least 80% sequence identity to the amino acid sequence S₂₁₁-L₂₂₅ of GR (SEQ ID NO: 4).

In yet another embodiment of the above method or methods, an amino acid residue of the polypeptide which corresponds to position S₂₁₁ of GR may comprise a wild-type (WT) residue, an S_{211A} mutant residue, or an S_{211E} mutant residue.

In yet another embodiment of the above method or methods, the agent may comprise a polypeptide having the amino acid sequence S₂₁₁-L₂₂₅ of GR (SEQ ID NO: 4), or at least any 7 consecutive amino acids thereof.

In still another embodiment of the above method or methods, the agent may comprise a mimic of a tetratricopeptide repeat (TPR) domain (FKBP51_{TPR}; SEQ ID NO: 6) of FKBP1 which interacts with GR in the GR-FKBP51 complex.

In another embodiment of the above method or methods, the agent may comprise a mimic of a FKBP51_{TPR3} region of FKBP51 (SEQ ID NO: 7).

In still another embodiment of the above method or methods, the agent may comprise a polypeptide having at least 80% sequence identity to the amino acid sequence of the FKBP51_{TPR3} region (SEQ ID NO: 7).

In another embodiment of the above method or methods, the agent may comprise a polypeptide having the amino acid sequence of the FKBP51_{TPR3} region (SEQ ID NO: 7), or at least any 7 consecutive amino acids thereof.

In still another embodiment of the above method or methods, the agent may further comprise a delivery moiety or delivery vehicle which facilitates translocation of the agent through a cell membrane and/or a blood brain barrier.

In another embodiment of the above method or methods, the delivery moiety may comprise a cell membrane transduction domain of a human immunodeficiency virus (HIV) transactivator of transcription (TAT) protein.

In still another embodiment of the above method or methods, the delivery moiety may comprise a cell-membrane transduction domain of HIV-type 1 TAT protein.

In yet another embodiment of the above method or methods, the delivery moiety may comprise an amino acid sequence of SEQ ID NO: 8, or a sequence having at least 80% sequence identity therewith.

In still another embodiment of the above method or methods, wherein the agent may comprise a polypeptide having at least 80% sequence identity to the amino acid sequence GRKKRRQRRRPQSPWRSDLLIDENCLL (SEQ ID NO: 9).

In yet another embodiment of the above method or methods, the agent may comprise a polypeptide having the amino acid sequence GRKKRRQRRRPQSPWRSDLLIDENCLL (SEQ ID NO: 9).

In another embodiment of the above method or methods, the method may further comprise the following step, performed before the step of administering:
measuring a level of GR-FKBP51 complex in the subject, comparing the measured level to a reference or control level representative of a non-PTSD condition, and identifying the subject as a candidate for treatment where the measured level of GR-FKBP51 complex in the subject is elevated relative to the reference or control level.

In yet another embodiment of the above method or methods, administration of the agent may normalize decreased GR phosphorylation levels, increase GR-FKBP52 complex levels, increase nuclear translocation of GR, or any combination thereof, in the subject.

In yet another embodiment of the above method or methods, the agent may be administered to the subject having elevated levels of GR-FKBP51 complex following a traumatic event, in order to prevent or reduce subsequent emergence of PTSD.

In yet another embodiment of the above method or methods, at least a portion of the agent may be administered to the brain of the subject, either directly, or indirectly via systemic or local administration to the subject followed by diffusion or transport to the brain.

In still another embodiment of the above method or methods, administration of the agent may block emergence of PTSD fear-related behaviours, inhibit the consolidation of cued fear memory, or both, in the aftermath of trauma.

In yet another embodiment of the above method or methods, administration of the agent may increase phosphorylation levels of position S₂₁₁ of GR in the subject.

In yet another embodiment, there is provided herein a use of an agent which inhibits formation of a glucocorticoid receptor (GR)-FK506 Binding Protein 51 (FKBP51) complex (GR-FKBP51 complex), or which disrupts already formed GR-FKBP51 complex, for preventing or treating post-traumatic stress disorder (PTSD) in a subject in need thereof, where the agent is for administration to the subject to reduce a level of GR-FKBP51 complex in the subject.

In another embodiment of the above use, the agent may comprise:
a competitive binder for a region of GR which binds FKBP51 in the GR-FKBP51 complex; or
a competitive binder for a region of FKBP51 which binds GR in the GR-FKBP51 complex.

In still another embodiment of the above use or uses, the agent may comprise a mimic of an N-terminal region of GR (GR_{NT}; SEQ ID NO: 2) which interacts with FKBP51 in the GR-FKBP51 complex.

In yet another embodiment of the above use or uses, the agent may comprise a mimic of a GR_{NT-4} (SEQ ID NO: 3) or GR_{NT-4-1} (SEQ ID NO: 4) region of GR.

In still another embodiment of the above use or uses, the agent may comprise a polypeptide having at least 80% sequence identity to the amino acid sequence S₂₁₁-L₂₂₅ of GR (SEQ ID NO: 4).

In another embodiment of the above use or uses, an amino acid residue of the polypeptide which corresponds to position S₂₁₁ of GR may comprise a wild-type (WT) residue, an S_{211A} mutant residue, or an S_{211E} mutant residue.

In still another embodiment of the above use or uses, the agent may comprise a polypeptide having the amino acid sequence S₂₁₁-L₂₂₅ of GR (SEQ ID NO: 4), or at least any 7 consecutive amino acids thereof.

In still another embodiment of the above use or uses, the agent may comprise a mimic of a tetratricopeptide repeat (TPR) domain (FKBP51_{TPR}; SEQ ID NO: 6) of FKBP1 which interacts with GR in the GR-FKBP51 complex.

In still another embodiment of the above use or uses, the agent may comprise a mimic of a FKBP51_{TPR3} region of FKBP51 (SEQ ID NO: 7).

In yet another embodiment of the above use or uses, the agent may comprise a polypeptide having at least 80% sequence identity to the amino acid sequence of the FKBP51_{TPR3} region (SEQ ID NO: 7).

In still another embodiment of the above use or uses, the agent may comprise a polypeptide having the amino acid sequence of the FKBP51_{TPR3} region (SEQ ID NO: 7), or at least any 7 consecutive amino acids thereof.

In still another embodiment of the above use or uses, the agent may further comprise a delivery moiety or delivery vehicle which facilitates translocation of the agent through a cell membrane and/or a blood brain barrier.

In still another embodiment of the above use or uses, the delivery moiety may comprise a cell membrane transduction domain of a human immunodeficiency virus (HIV) transactivator of transcription (TAT) protein.

In still another embodiment of the above use or uses, the delivery moiety may comprise a cell-membrane transduction domain of HIV-type 1 TAT protein.

In still another embodiment of the above use or uses, the delivery moiety may comprise an amino acid sequence of SEQ ID NO: 8, or a sequence having at least 80% sequence identity therewith.

In yet another embodiment of the above use or uses, the agent may comprise a polypeptide having at least 80% sequence identity to the amino acid sequence GRKKRRQRRRPQSPWRSDLLIDENCLL (SEQ ID NO: 9).

In still another embodiment of the above use or uses, the agent may comprise a polypeptide having the amino acid sequence GRKKRRQRRRPQSPWRSDLLIDENCLL (SEQ ID NO: 9).

In another embodiment of the above use or uses, the subject may be a subject having a measured level of GR-FKBP51 complex which is elevated relative to a reference or control level representative of a non-PTSD condition.

In yet another embodiment of the above use or uses, the agent may be for normalizing decreased GR phosphorylation levels, increasing GR-FKBP52 complex levels, increasing nuclear translocation of GR, or any combination thereof, in the subject.

In still another embodiment of the above use or uses, the agent may be for administration to the subject having elevated levels of GR-FKBP51 complex following a traumatic event, in order to prevent or reduce subsequent emergence of PTSD.

In another embodiment of the above use or uses, at least a portion of the agent may be for administration to the amygdala of the subject, either directly, or indirectly via systemic or local administration to the subject followed by diffusion or transport to the amygdala.

In another embodiment of the above use or uses, the agent may be for blocking emergence of PTSD fear-related behaviours, inhibiting the consolidation of cued fear memory, or both, in the aftermath of trauma.

In still another embodiment of the above use or uses, the agent may be for increasing phosphorylation levels of position S₂₁₁ of GR in the subject.

In still another embodiment, there is provided herein a method for diagnosing a subject as having, or being at risk of developing, a post-traumatic stress disorder (PTSD), said method comprising:
measuring a level of a glucocorticoid receptor (GR)-FK506 Binding Protein 51 (FKBP51) complex (GR-FKBP51 complex) in the subject;
comparing the measured level to a reference or control level representative of a non-PTSD condition; and
identifying the subject as having, or being at risk of developing, PTSD where the measured level of GR-FKBP51 complex in the subject is elevated relative to the reference or control level.

In another embodiment of the above method, the subject may be a subject who has experienced, or is at risk of experiencing, a severe psychological trauma.

In another embodiment of the above method or methods, the method may further comprise a step of treating the subject for PTSD where the subject is identified as having, or being at risk of developing, PTSD. In certain embodiments, the subject may be treated using any suitable conventional treatment for PTSD, using therapy or counselling, using a PTSD pharmaceutical treatment, using a method for treating PTSD as described herein, or any combination thereof.

In yet another embodiment of the above method or methods, the step of measuring may be performed using a sample of peripheral blood, a sample of lymphocytes, or other blood sample, obtained from the subject.

In yet another embodiment of the above method or methods, the step of measuring may comprise ELISA, co-immunoprecipitation, and/or Western blotting to quantify the level of GR-FKBP51 complex.

In still another embodiment of the above method or methods, the method may further comprise one or more of: measuring a level of GR S₂₁₁ phosphorylation in the subject, where a decreased level relative to a reference or control level representative of a non-PTSD condition is further indicative of the subject having, or being at risk of developing, PTSD; measuring a level of GR-FKBP52 complex in the subject, where a decreased level relative to a reference or control level representative of a non-PTSD condition is further indicative of the subject having, or being at risk of developing, PTSD; or measuring a level of nuclear GR in the subject, where a decreased level relative to a reference or control level representative of a non-PTSD condition is further indicative of the subject having, or being at risk of developing, PTSD.

In yet another embodiment of the above method or methods, the subject identified as having, or being at risk of developing, PTSD may be further identified as a candidate for treatment by a method as defined herein.

In still another embodiment, there is provided herein a use of a glucocorticoid receptor (GR)-FK506 Binding Protein 51 (FKBP51) complex (GR-FKBP51 complex) for diagnosing a subject as having, or being at risk of developing, a post-traumatic stress disorder (PTSD), wherein a measured level of the GR-FKBP51 complex in the subject is indicative of the subject having, or being at risk of developing, PTSD when the measured level is elevated relative to a reference or control level representative of a non-PTSD condition.

In another embodiment of the above use, the subject may be a subject who has experienced, or is at risk of experiencing, a severe psychological trauma.

In still another embodiment of the above use or uses, the measured level may be determined using a sample of peripheral blood, a sample of lymphocytes, or other blood sample, from the subject.

In yet another embodiment of the above use or uses, the measured level may be determined using ELISA, co-immunoprecipitation, and/or Western blotting to quantify the level of GR-FKBP51 complex.

In another embodiment of the above use or uses, a decreased level of GR S₂₁₁ phosphorylation in the subject; a decreased level of GR-FKBP52 complex in the subject; a decreased level of nuclear GR in the subject; or any combination thereof, relative to a reference or control level representative of a non-PTSD condition may be further indicative of the subject having, or being at risk of developing, PTSD.

In still another embodiment of the above use or uses, the subject identified as having, or being at risk of developing, PTSD may be further identified as a candidate for treatment with an agent which inhibits formation of the GR-FKBP51 complex, or which disrupts already formed GR-FKBP51 complex.

In another embodiment, there is provided herein an agent which inhibits formation of a glucocorticoid receptor (GR)-FK506 Binding Protein 51 (FKBP51) complex (GR-FKBP51 complex), or which disrupts already formed GR-FKBP51 complex.

In another embodiment of the above agent, the agent may comprise:
a competitive binder for a region of GR which binds FKBP51 in the GR-FKBP51 complex; or
a competitive binder for a region of FKBP51 which binds GR in the GR-FKBP51 complex.

In yet another embodiment of the above agent or agents, the agent may comprise a mimic of an N-terminal region of GR (GR_{NT}; SEQ ID NO: 2) which interacts with FKBP51 in the GR-FKBP51 complex.

In still another embodiment of the above agent or agents, the agent may comprise a mimic of a GR_{NT-4} (SEQ ID NO: 3) or GR_{NT-4-1} (SEQ ID NO: 4) region of GR.

In yet another embodiment of the above agent or agents, the agent may comprise a mimic of a tetratricopeptide repeat (TPR) domain (FKBP51_{TPR}; SEQ ID NO: 6) of FKBP1 which interacts with GR in the GR-FKBP51 complex.

In still another embodiment of the above agent or agents, the agent may comprise a mimic of a FKBP51_{TPR3} region of FKBP51 (SEQ ID NO: 7).

In another embodiment of the above agent or agents, the agent may further comprise a delivery moiety or delivery vehicle which facilitates translocation of the agent through a cell membrane and/or a blood brain barrier.

In yet another embodiment of the above agent or agents, the delivery moiety may comprise a cell membrane transduction domain of a human immunodeficiency virus (HIV) transactivator of transcription (TAT) protein.

In yet another embodiment of the above agent or agents, the delivery moiety may comprise a cell-membrane transduction domain of HIV-type 1 TAT protein.

In still another embodiment of the above agent or agents, the delivery moiety may comprise an amino acid sequence of SEQ ID NO: 8, or a sequence having at least 80% sequence identity therewith.

In still another embodiment, there is provided herein a polypeptide comprising a polypeptide sequence having at least 80% sequence identity to the amino acid sequence S₂₁₁-L₂₂₅ of GR (SEQ ID NO: 4).

In another embodiment of the above polypeptide, an amino acid residue of the polypeptide which corresponds to position S₂₁₁ of GR may comprise a wild-type (WT) residue, an S_{211A} mutant residue, or an S_{211E} mutant residue.

In still another embodiment of the above polypeptide or polypeptides, the polypeptide may comprise a polypeptide sequence having the amino acid sequence S₂₁₁-L₂₂₅ of GR (SEQ ID NO: 4), or at least any 7 consecutive amino acids thereof.

In still another embodiment of the above polypeptide or polypeptides, the polypeptide may further comprise a delivery moiety or delivery vehicle which facilitates translocation of the agent through a cell membrane and/or a blood brain barrier.

In still another embodiment of the above polypeptide or polypeptides, the delivery moiety may comprise a cell membrane transduction domain of a human immunodeficiency virus (HIV) transactivator of transcription (TAT) protein.

In yet another embodiment of the above polypeptide or polypeptides, the delivery moiety may comprise a cell-membrane transduction domain of HIV-type 1 TAT protein.

In another embodiment of the above polypeptide or polypeptides, the delivery moiety may comprise an amino acid sequence of SEQ ID NO: 8, or a sequence having at least 80% sequence identity therewith.

In still another embodiment of the above polypeptide or polypeptides, the polypeptide may comprise a polypeptide sequence having at least 80% sequence identity to the amino acid sequence GRKKRRQRRRPQSPWRSDLLIDENCLL (SEQ ID NO: 9).

In yet another embodiment of the above polypeptide or polypeptides, the polypeptide may comprise a polypeptide sequence having the amino acid sequence GRKKRRQRRRPQSPWRSDLLIDENCLL (SEQ ID NO: 9).

In another embodiment, there is provided herein a polypeptide comprising a polypeptide sequence having at least 80% sequence identity to the amino acid sequence of the FKBP51_{TPR3} region (SEQ ID NO: 7).

In another embodiment of the above polypeptide, the polypeptide may comprise a polypeptide sequence having the amino acid sequence of the FKBP51_{TPR3} region (SEQ ID NO: 7), or at least any 7 consecutive amino acids thereof.

In still another embodiment of the above polypeptide or polypeptides, the polypeptide may further comprise a delivery moiety or delivery vehicle which facilitates translocation of the agent through a cell membrane and/or a blood brain barrier.

In still another embodiment of the above polypeptide or polypeptides, the delivery moiety may comprise a cell membrane transduction domain of a human immunodeficiency virus (HIV) transactivator of transcription (TAT) protein.

In another embodiment of the above polypeptide or polypeptides, the delivery moiety may comprise a cell-membrane transduction domain of HIV-type 1 TAT protein.

In yet another embodiment of the above polypeptide or polypeptides, the delivery moiety may comprise an amino acid sequence of SEQ ID NO: 8, or a sequence having at least 80% sequence identity therewith.

In another embodiment, there is provided herein a pharmaceutical composition comprising the agent of any one of claims 59-68, the polypeptide of any one of claims 69-83, or any combination thereof, and a pharmaceutically acceptable excipient, diluent, or carrier.

In still another embodiment of the above pharmaceutical composition, the pharmaceutical composition may further comprise a delivery moiety and/or a delivery vehicle.

In yet another embodiment, the delivery moiety or delivery vehicle may comprise a targeting moiety, a translocation moiety, a nanoparticle, or a nanovesicle configured for delivery of the pharmaceutical composition to the brain.

In another embodiment, there is provided herein a kit for diagnosing PTSD, said kit comprising at least one of.
an anti-glucocorticoid receptor (GR) antibody;
an anti-FK506 Binding Protein 51 (FKBP51) antibody;
an anti-GR-FKBP51 complex antibody; or
instructions for using the kit to perform a method as described herein.

In another embodiment, there is provided herein an ELISA or other immunoassay kit for measuring a level of GR-FKBP51 complex in a sample from a subject, or for diagnosing PTSD, the kit comprising at least one of.
an anti-FKBP51 primary antibody;
an anti-GR primary antibody;
a secondary antibody capable of binding to one of the primary antibodies, wherein the secondary antibody is, optionally, labelled or conjugated with an enzyme for fluorescent or chemiluminescent detection;
an ECL (enhanced chemiluminescent), or other fluorescent or chemiluminescent, substrate;
BSA or a blocking agent;
a wash buffer; and/or
instructions for using the kit to measure the level of GR-FKBP51 complex in the sample from the subject, or for diagnosing PTSD.

In another embodiment of the above kit, the anti-FKBP51 antibody, the anti-GR antibody, or both, may be immobilized or covalently bound to a surface or membrane.

In another embodiment, there is provided herein a method for diagnosing a subject as having, or being at risk of developing, a post-traumatic stress disorder (PTSD), said method comprising:
measuring a level of a glucocorticoid receptor (GR)-FK506 Binding Protein 51 (FKBP51) complex (GR-FKBP51 complex) in a sample from the subject by ELISA assay using anti-FKBP51 antibody for complex pull down and anti-GR antibody for complex detection;
comparing the measured level to a reference or control level representative of a non-PTSD condition; and
identifying the subject as having, or being at risk of developing, PTSD where the measured level of GR-FKBP51 complex in the subject is elevated relative to the reference or control level.

In still another embodiment, there is provided herein a method for diagnosing a subject as having, or being at risk of developing, a post-traumatic stress disorder (PTSD), said method comprising:
measuring a level of a glucocorticoid receptor (GR)-FK506 Binding Protein 51 (FKBP51) complex (GR-FKBP51 complex) in a sample from the subject by ELISA assay using anti-GR antibody for complex pull down and anti-FKBP51 antibody for complex detection;
comparing the measured level to a reference or control level representative of a non-PTSD condition; and
identifying the subject as having, or being at risk of developing, PTSD where the measured level of GR-FKBP51 complex in the subject is elevated relative to the reference or control level.

In another embodiment of the method or methods described above, the subject may be identified as having, or being at risk of developing, PTSD where the measured level of GR-FKBP51 complex in the subject is elevated relative to the reference or control level by about 1.1 fold or greater; about 1.2 fold or greater; about 1.3 fold or greater; about 1.4 fold or greater; about 1.5 fold or greater; about 1.6 fold or greater; about 1.7 fold or greater; about 1.8 fold or greater; about 1.9 fold or greater; or about 2.0 fold or greater.

In another embodiment of the method or methods above, the subject may be identified as having, or being at risk of developing, PTSD where the measured level of GR-FKBP51 complex in the subject is elevated relative to the reference or control level by about 1.40 fold or greater; about 1.41 folder or greater; about 1.42 fold or greater; about 1.43 fold or greater; about 1.44 fold or greater; about 1.45 fold or greater; about 1.46 fold or greater; about 1.47 fold or greater; about 1.48 fold or greater; about 1.49 fold or greater; about 1.50 fold or greater; about 1.51 fold or greater; about 1.52 fold or greater; about 1.53 fold or greater; about 1.54 fold or greater; about 1.55 fold or greater; about 1.56 fold or greater; about 1.57 fold or greater; about 1.58 fold or greater; about 1.59 fold or greater; or about 1.60 fold or greater.

In another embodiment, there is provided herein a method for identifying PTSD therapeutic candidates, said method comprising:
exposing one or more candidate agents to a mixture of glucocorticoid receptor (GR) and FK506 Binding Protein 51 (FKBP51) under conditions in which GR and FKBP51 are able to form GR-FKBP51 complex; and
measuring a level of GR-FKBP51 complex formed in the presence of the one or more candidate agents;
wherein a reduction in the level of GR-FKBP51 complex formed in the presence of the one or more candidate agents, as compared to a corresponding reference level of GR-FKBP51 complex formed in the absence of the one or more candidate agents, identifies the one or more candidate agents as PTSD therapeutic candidates.

In still another embodiment, there is provided herein a method for identifying PTSD therapeutic candidates, said method comprising:
exposing one or more candidate agents to a mixture of glucocorticoid receptor (GR) and FK506 Binding Protein 51 (FKBP51) under conditions in which GR and FKBP51 are able to form GR-FKBP51 complex; and
determining whether GR-FKBP51 complex levels are reduced by the one or more candidate agents using bioluminescence resonance energy transfer (BRET), fluorescence resonance energy transfer (FRET), or surface plasmon resonance (SPR)-based detection;
wherein a reduction in the level of GR-FKBP51 complex formed in the presence of the one or more candidate agents, as compared to a corresponding reference level of GR-FKBP51 complex formed in the absence of the one or more candidate agents, identifies the one or more candidate agents as PTSD therapeutic candidates.

### BRIEF DESCRIPTION OF DRAWINGS

FIG. 1 shows that GR-FKBP51 complex levels were significantly higher in peripheral blood samples from PTSD patients. A. In mouse brain lysate, GR antibody, but not IgG (negative control), co-immunoprecipitated with FKBP51. B. In mouse brain lysate, FKBP51 antibody, but not IgG (negative control), co-immunoprecipitated with GR. C. Co-immunoprecipitation shows higher levels of the GR-FKBP51 complex in fear-conditioned mouse brain lysate as compared to control (CTRL) mice. D. Densitometric analysis of the levels of FKBP51 co-immunoprecipitated by GR antibody in brain lysate of control (CTRL) or fear-conditioned mice. The level of co-immunoprecipitated FKBP51 (FKBP51 Co-IP) was normalized after being divided by the level of precipitated GR (GR IP). Results for each sample are presented as the percentage of control (CTRL). **p<0.01 as compared to control samples, n=3, t-test. Data was shown as mean±S.E.M. E-G. Co-immunoprecipitation shows significantly more GR-FKBP51 complex in peripheral blood samples from PTSD patients compared to controls. E. Representative western blot of GR and FKBP51 precipitated by FKBP51 antibody. The intensity of each protein band for GR or FKBP51 was quantified by densitometry. Results for each sample are presented as the percentage of the mean of the control samples on the same blot. F. Co-immunoprecipitation shows significantly more GR-FKBP51 complex in peripheral blood samples from PTSD patients compared to healthy controls. (CTRL: 1±0.069, PTSD: 1.408±0.641, *** p<0.001, n=22, t-test). G. Co-immunoprecipitation shows significantly more GR-FKBP51 complex in peripheral blood samples from PTSD patients compared to trauma controls (Trauma: 1±0.061, PTSD: 1.455±0.661, *** p<0.001, n=21, t-test);

FIG. 2 shows that GR forms a complex with FKBP51 via the S211-L225 region of the amino-terminus of GR. A. Western blot showing that GST-GR_{NT}, but not GST-GR_{CT}, can “pull-down” FKBP51 in mouse brain tissue. B. Western blot showing that GST-GR_{NT4}, but not GST-GR_{NT1}, GST-GR_{NT2}, GST-GR_{NT3}, GST-GR_{NT5} or GST-GR_{NT6} can “pull-down” FKBP51 in mouse brain tissue. C. Western blot showing that GST-GR_{NT4-1}, but not GST-GR_{NT4-2}, GST-GR_{NT4-3}, GST-GR_{NT4-4} or GST-GR_{NT4-5}, can “pull-down” of FKBP51 in mouse brain tissue. D. Western blot showing that GST-TPR, but not GST-FK1 or GST-FK2, can “pull-down” GR in mouse brain tissue. E. Western blot showing that GST-TPR3, but not GST-TPR1, GST-TPR2 or GST-TPR4 can “pull-down” GR in mouse brain tissue. F. Co-immunoprecipitation shows that TAT-GRpep, but not TAT, was able to disrupt the GR-FKBP51 complex in mouse brain slices. G. TAT-GRpep specifically disrupted the interaction between GR and FKBP51. The TAT-GRpep peptide is composed of the part of the GR sequence where it binds to FKBP51, and the HIV transactivator of transcription (TAT) which facilitates the translocation of this fusion peptide through blood brain barriers and cell membranes. In the presence of TAT-GRpep, it competes with GR in binding to FKBP51, resulting in more GR in its non-binding form;

FIG. 3 shows that administration of TAT-GRpep into amygdala reduced fear memory. A. A schematic illustration of the experimental schedule. B. The two group did not differ in the acquisition of cued-fear memory (two-way ANOVA with repeated measures, F1,21=0.25, p=NS). C. Two-way ANOVA confirmed significant cue effect (F1, 46=10.387, p<0.01), and treatment effect (F1, 46=7.708, p<0.01). The fear memory assessment showed that the treatment peptide (TAT-GRpep) alleviated cued fear memory as the animals injected with TAT-GRpep showed significantly lower level of freezing behavior compared to their control counterparts (p<0.01). D. Two groups did not differ in the acquisition of cued-fear memory (two-way ANOVA with repeated measures (p=NS). E. Two-way ANOVA confirmed no significant treatment effect (F1, 20=0.353, p=NS). Both treatment groups do not differ in freezing behaviour whether the cue was on (p=NS) or off (p=NS);

FIG. 4 shows that administration of TAT-GRpep systemically reduced freezing behaviours. A. A schematic illustration of the experimental schedule regarding Figure B. B. During the three-minute habituation (absence of CS), the animals from both groups displayed a virtual absence of freezing behavior (p=NS). When cues were presented, however, animals from the treatment peptide group manifested a lower level of freezing behavior compared to the control peptide (TAT) group (p<0.05). C. A schematic illustration of the experimental schedule regarding Figure D. D. TAT-GRpep reduced cued freezing behaviours in mice (p<0.05). E. Co-immunoprecipitation shows that TAT-GRpep, but not TAT, was able to block the increase of GR-FKBP51 complex in fear-conditioned mice. F. Densitometric analysis of the level of FKBP51 co-immunoprecipitated by GR antibody in brain lysate of fear-conditioned mice injected with TAT, or TAT-GRpep. The level of co-immunoprecipitated FKBP51 (FKBP51 Co-IP) was normalized after being divided by the level of precipitated GR (GR IP). Results for each sample are presented as the percentage of fear conditioning group. *p<0.05 as compared to fear conditioning samples, n=3, one-way ANOVA test followed by Dunnett post hoc test. Data was shown as mean±S.E.M. G. Co-immunoprecipitation shows that TAT-GRpep was able to disrupt the GR-FKBP51 complex in lymphocyte lysate from blood of fear-conditioned mice. H. Densitometric analysis of the level of FKBP51 co-immunoprecipitated by GR antibody in lymphocyte lysate of fear-conditioned mice injected with TAT, or TAT-GRpep. The level of co-immunoprecipitated FKBP51 (FKBP51 Co-IP) was normalized after being divided by the level of precipitated GR (GR IP). Results for each sample are presented as the percentage of TAT+fear conditioning group. **p<0.01 as compared to TAT+fear conditioning samples, n=4, t-test. Data was shown as mean±S.E.M.;

FIG. 5 shows that GR phosphorylation at S₂₁₁ was decreased in lymphocytes from PTSD patients, which induces the binding affinity of GR to FKBP51. A. Western blot shows lower levels of GR phosphorylation at S₂₁₁ in mouse brain lysate of fear-conditioned mice as compared to control (CTRL) mice. GR was used as a loading control. B. Densitometric analysis of the levels of GR phosphorylation at S₂₁₁ in mouse brain lysate of fear-conditioned mice or control (CTRL) mice. The level of phosphorylated GR (S₂₁₁) was normalized after being divided by the level of GR. Results for each sample are presented as the percentage of the control (CTRL) sample. *p<0.05, n=3, t-test. Data was shown as mean±S.E.M. C. Western blot analysis shows a decreased GR phosphorylation at S₂₁₁ in lymphocytes from PTSD patients compared to healthy controls (CTRL). (* p<0.05, n=12, t-test). D. Co-immunoprecipitation shows higher levels of the GR-FKBP51 complex in HEK293T cells transfected with S_{211A}-GR, as compared to wild-type (WT) GR, while there is no significant difference between S_{211E}-GR and WT-GR. E. Densitometric analysis of the level of Flag-GR co-immunoprecipitated by GFP-FKBP51 antibody in HEK293T cells transfected with WT-GR, S_{211A}-GR, or S_{211E}-GR as well as GFP-FKBP51. The level of co-immunoprecipitated Flag-GR (Flag-GR Co-IP) was normalized after being divided by the level of precipitated GFP-FKBP51 (GFP-FKBP51 IP). Results for each sample are presented as the percentage of wild-type (WT) group on the same blot. *p<0.05 as compared to WT samples, n=3, one-way ANOVA test followed by Dunnett post hoc test. Data was shown as mean±S.E.M. F-H. Flag-tagged GR (wild-type, S_{211A}, S_{211E}) was immobilized on the sensor chip for SPR kinetic binding analysis with FKBP51-TPR. F. GR (WT) binds FKBP51-TPR with K_{D}=3.31(4) uM. G. non-phosphorylated GR mutant S_{211A} binds FKBP51 with higher affinity with K_{D}=412(7) nM. H. phosphomimetic GR mutant S_{211E} binds FKBP51 with similar affinity with K_{D}=3.90(1) uM. I. Western blot shows that TAT-GRpep, but not TAT, is able to block the decrease of GR phosphorylation at S₂₁₁ in mouse brain lysate of fear-conditioned mice. GR was used as a loading control. J. Densitometric analysis of the levels of GR phosphorylation at S₂₁₁ in mouse brain lysate of fear-conditioned mice injected with TAT or TAT-GRpep. The level of phosphorylated GR (S₂₁₁) was normalized after being divided by the level of GR. Results for each sample are presented as the percentage of fear conditioning sample. *p<0.05, n=3, one-way ANOVA test followed by Dunnett post hoc test. Data was shown as mean±S.E.M. K. Proposed model/summary of potential molecular pathways within the neuron in the presence of TAT-GRpep. 1) TAT-GRpep may be able to pass through the blood brain barrier and enter into the neuron due to fusion with the TAT sequence. 2) Once TAT-GRpep enters the cell, it may compete with GR in binding to FKBP51, which may result in more GR in its non-binding form. 3) Consequently, more GR may bind to FKBP52, and 4) may undergo phosphorylation by kinases. 5) Both events may be responsible for translocating GR into the nucleus, where it may bind to specific DNA sequence and may promote transcription;

FIG. 6 shows that disruption of GR-FKBP51 complex resulted in GR-FKBP52 binding and GR nuclear translocation in vitro. A. Western blot analysis shows more FKBP52 was co-immunoprecipitated by GR antibody in protein extract from mouse brain slices treated with TAT-GRpep (10 μM, 30 min) compared to those from untreated controls (CTRL) or those treated with TAT alone. B. Densitometric analysis of the levels of FKBP52 co-immunoprecipitated by GR antibody in mouse brain slices with TAT-GRpep, TAT or untreated controls (CTRL). The level of co-immunoprecipitated FKBP52 (FKBP52 Co-IP) was normalized after being divided by the level of precipitated GR (GR IP). Results for each sample are presented as the percentage of control (CTRL) on the same blot. *p<0.05 as compared to the control (CTRL) sample, n=3, one-way ANOVA test followed by Dunnett post hoc test. Data was shown as mean±S.E.M. C. Western blot analysis shows higher levels of nuclear GR in mouse brain slices treated with TAT-GRpep (10 μM, 30 min) compared to TAT or untreated controls (CTRL). Histone H3 was used as loading control. D. Densitomeric analysis of the levels of nuclear expression of GR in mouse brain slices. The level of nuclear expression of GR was normalized after being divided by the level of Histone H3. Results for each sample are presented as the percentage of the control sample (CTRL). ***p<0.001 as compared to the control sample (CTRL), n=3, one-way ANOVA test followed by Dunnett post hoc test. Data was shown as mean±S.E.M. E. TAT-GRpep treatment resulted in more GR and FKBP52 colocalization within the nucleus of primary neurons. Immunocytochemistry was performed on primary neurons at 8 days in vitro (DIV) with antibodies against glucocorticoid receptors (GR) (green) and FK506-binding protein 52 (FKBP52) (red). DAPI was used as a counterstain to label nuclei. Primary neurons were treated with either saline, 10 μM TAT-control peptide or 10 μM TAT-GRpep for 45 min. TAT-GRpep-treated neurons displayed significantly more colocalization between GR and FKBP52 when compared to saline and control peptide groups. In addition, almost negligible amount of GR-FKBP52 complex were observed in the nuclei of neurons with saline and control peptide treatment, but TAT-GRpep administration resulted in a marked increase in nuclear GR-FKBP52 colocalization (white arrows), suggesting that blocking this interaction may affect both GR and FKBP52 complex formation and their nuclear localization;

FIG. 7 shows that disruption of GR-FKBP51 complex resulted in GR-FKBP52 binding and GR nuclear translocation in fear-conditioned animal and PTSD patients. A. Western blot analysis shows that more FKBP52 was co-immunoprecipitated by GR antibody in protein extract from fear-conditioned mouse brain injected with TAT-GRpep (3 nmol/g i.p) compared to those injected with TAT alone or saline. B. Densitometric analysis of the levels of FKBP52 co-immunoprecipitated by GR antibody in fear-conditioned mice injected with TAT-GRpep or TAT peptide. The intensity of each protein band for FKBP52 and GR was quantified by densitometry. The level of co-immunoprecipitated FKBP52 (FKBP52 Co-IP) was normalized after being divided by the level of precipitated GR (GR IP). Results for each sample are presented as the percentage of the fear-conditioned sample. *p<0.05 as compared to the fear conditioning sample, n=3, one-way ANOVA test followed by Dunnett post hoc test. Data was shown as mean±S.E.M. C. Western blot analysis shows higher levels of nuclear GR from fear-conditioned mice injected with TAT-GRpep (3 nmol/g i.p) compared to those injected with TAT alone or saline. Histone H3 was used as loading control. D. Densitometric analysis of nuclear expression of GR in fear-conditioned mice injected with TAT-GRpep (3 nmol/g i.p) compared to those injected with TAT alone or saline. Densitometric analysis of nuclear expression of GR in mouse brain tissue. The level of nuclear expression of GR was normalized after being divided by the level of Histone H3. Results for each sample are presented as the percentage of the fear-conditioned sample. *p<0.05 as compared to the fear-conditioned group, n=4, one-way ANOVA test followed by Dunnett post hoc test. Data was shown as mean±S.E.M. E. Densitometric analysis of nuclear expression of GR in lymphocytes from PTSD patients compared to healthy controls (CTRL). (** p<0.01, n=12, t-test). F. Representative western blot of 14-3-3 ε and Actin expression in lymphocytes from PTSD patients compared to healthy controls (CTRL). The intensity of each protein band was quantified by densitometry. G. Results (Mean±SEM) are presented as the percentage of the mean of the control samples on the same plot. (* p<0.05, n=12, t-test). H. FKBP51 binding inhibits nuclear translocation of GR. Reduced nuclear translocation of GR is negatively correlated with higher GR-FKBP51 complex levels in lymphocytes from PTSD patients compared to CTRL. [n=24 (12 PTSD and 12 CTRL), Pearson correlation coefficient r=−0.44392, p=0.03];

FIG. 8 shows a schematic representation of generated GST-fusion proteins encoding truncated GR segments used for identifying the region of GR which interacts with FKBP51 in the GR-FKBP51 complex;

FIG. 9 shows a schematic representation of generated GST-fusion proteins encoding truncated FKBP51 segments used for identifying the region of FKBP51 which interacts with GR in the GR-FKBP51 complex; and

FIG. 10 shows sequences of certain polypeptides which are described herein.

### DETAILED DESCRIPTION

Described herein are methods for preventing or treating post-traumatic stress disorder (PTSD) in a subject; methods for diagnosing a subject as having, or being at risk of developing, a PTSD; as well as agents, compositions, and/or kits for the diagnosis and/or treatment of PTSD. It will be appreciated that embodiments and examples are provided for illustrative purposes intended for those skilled in the art, and are not meant to be limiting in any way.

Post-traumatic stress disorder (PTSD) may develop after exposure to severe psychological trauma, leaving patients with disabling anxiety, nightmares and flashbacks. Traditional treatments are only partially effective, and development of better treatments has been hampered by limited knowledge of molecular mechanisms underlying PTSD. It has now been identified that the glucocorticoid receptor (GR) and FKBP51 (FK506 Binding Protein 51) form a protein complex that is elevated in fear-conditioned mice, an animal model for PTSD, and in PTSD patients compared to unaffected control subjects as well as subjects exposed to trauma without PTSD. Further, a peptide-based inhibitor has been developed that disrupts GR-FKBP51 binding, and in fear-conditioned mice, reduces freezing time. The peptide also normalized decreased GR phosphorylation, and increased both GR-FKBP52 binding and nuclear translocation of the GR in fear-conditioned mice. These developments may provide for methods of preventing or treating PTSD, methods for diagnosing PTSD, and/or agents, compositions, and/or kits therefor. A molecular mechanism contributing to PTSD is identified here in what is believed to be for the first time, and applied to the development of therapeutic and/or diagnostic approaches for PTSD.

Treatment of PTSD:

In certain embodiments, there is provided herein a method for preventing or treating post-traumatic stress disorder (PTSD) in a subject in need thereof, said method comprising:
administering an agent which inhibits formation of a glucocorticoid receptor (GR)-FK506 Binding Protein 51 (FKBP51) complex (GR-FKBP51 complex), or which disrupts already formed GR-FKBP51 complex, to the subject;

thereby reducing a level of GR-FKBP51 complex in the subject and preventing or treating the PTSD.

In another embodiment, there is provided herein a use of an agent which inhibits formation of a glucocorticoid receptor (GR)-FK506 Binding Protein 51 (FKBP51) complex (GR-FKBP51 complex), or which disrupts already formed GR-FKBP51 complex, for preventing or treating post-traumatic stress disorder (PTSD) in a subject in need thereof, where the agent is for administration to the subject to reduce a level of GR-FKBP51 complex in the subject.

As will be understood, in certain embodiments the method or use may be for preventing PTSD, which may include either preventing onset of PTSD, or reducing the severity of onset of one or more symptoms thereof. In certain embodiments, the subject may be treated by the method or use shortly following a traumatic event, and before onset of PTSD. In certain embodiments, the subject may be a subject at risk of experiencing a traumatic event, and the subject may be treated by the method as a precautionary measure.

In certain embodiments, the method or use may be for treating PTSD, which may include either treating or reducing the severity of PTSD, or at least one symptom thereof, in a subject in need thereof. In certain embodiments, the subject may be a subject at the early stages of PTSD onset, or may be a subject already living with PTSD, for example.

Accordingly, in certain embodiments, treatment may be preventative. In certain embodiments, treatment may avoid the development of PTSD symptoms, or may reduce their severity.

In certain embodiments, the agent which inhibits formation of the GR-FKBP51 complex, or which disrupts already formed GR-FKBP51 complex, may be any suitable agent which is able to reduce levels of GR-FKBP51 complex in the subject. In certain embodiments, the agent may include, for example, an agent which binds to GR, or which binds to FKBP51, so as to prevent GR and FKBP51 from binding each other to form the GR-FKBP51 complex. In certain embodiments, the agent may include, for example, an agent which binds to GR, or which binds to FKBP51, so as to disrupt already formed GR-FKBP51 complex, causing the complex to separate. In certain embodiments, the agent may comprise an agent which binds, blocks, or alters the structure of a region of GR which binds FKBP51 in the GR-FKBP51 complex, or which binds, blocks, or alters the structure of a region of FKBP51 which binds GR in the GR-FKBP51 complex, so as to reduce levels of GR-FKBP51 complex in the subject. In certain embodiments, for example, the agent may be an agent which binds directly to a portion of GR which interacts with FKBP51 in the GR-FKBP51 complex, or may be an agent which binds directly to a portion of FKBP51 which interacts with GR in the GR-FKBP51 complex. In certain embodiments, the agent may be a peptide-based agent, although various other types of agents are also contemplated such as, for example, a small molecule-based agent, or a nucleic acid-based agent (such as, for example, an aptamer). In certain embodiments, the agent may comprise an antibody or antibody fragment which binds to GR or FKBP51, or which binds to an epitope found in the region of GR or FKBP51 which is involved in forming the GR-FKBP51 complex, for example.

As described in further detail hereinbelow, it is the N-terminal region of GR (GR_{NT}) which may interact with FKBP51 in the GR-FKBP51 complex, and the tetratricopeptide repeat (TPR) domain (FKBP51_{TPR}) of FKBP1 which may interact with GR in the GR-FKBP51 complex. Accordingly, in certain embodiments, the agent may comprise an agent which binds, blocks, or alters the structure of the N-terminal region of GR (GR_{NT}) which interacts with FKBP51, or which binds, blocks, or alters the structure of the tetratricopeptide repeat (TPR) domain (FKBP51_{TPR}) of FKBP1 which interacts with GR.

In certain embodiments, the agent may comprise an agent which binds directly to a region of GR which otherwise interacts with FKBP1 in the GR-FKBP51 complex, or may comprise an agent which binds directly to a region of FKBP1 which otherwise interacts with GR in the GR-FKBP51 complex, thus reducing levels of GR-FKBP51 complex in the subject. Although direct binding to the interacting region is a preferred approach described herein, it is also contemplated that other approaches may be used, such as an allosteric-type binder which binds GR or FKBP51 elsewhere and still prevents formation or disrupts existing GR-FKBP51 complex, for example. In certain embodiments, it is contemplated that the agent may comprise a decoy or mimic of GR which binds FKBP51, or a decoy or mimic of FKBP51 which binds GR, and which reduces levels of GR-FKBP51 complex in the subject as a result.

In certain embodiments, the agent may comprise a competitive binder for a region of GR which binds FKBP51 in the GR-FKBP51 complex; or a competitive binder for a region of FKBP51 which binds GR in the GR-FKBP51 complex.

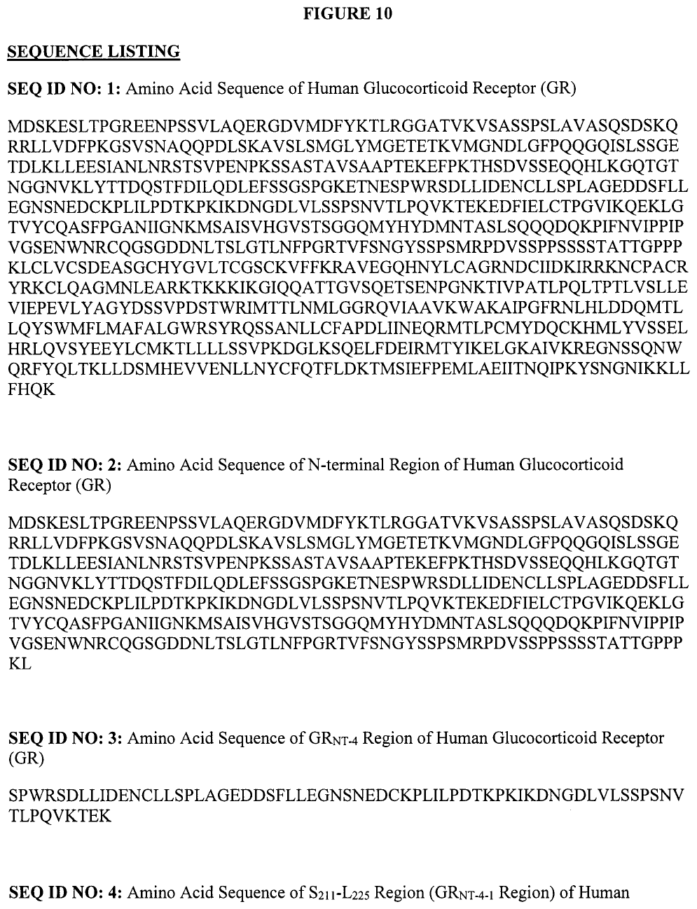

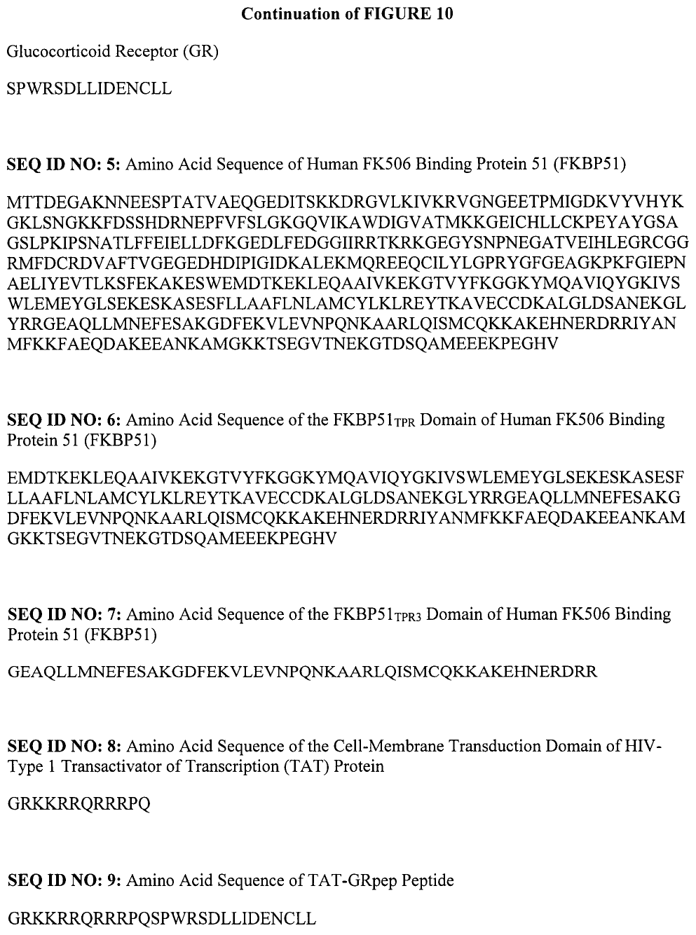

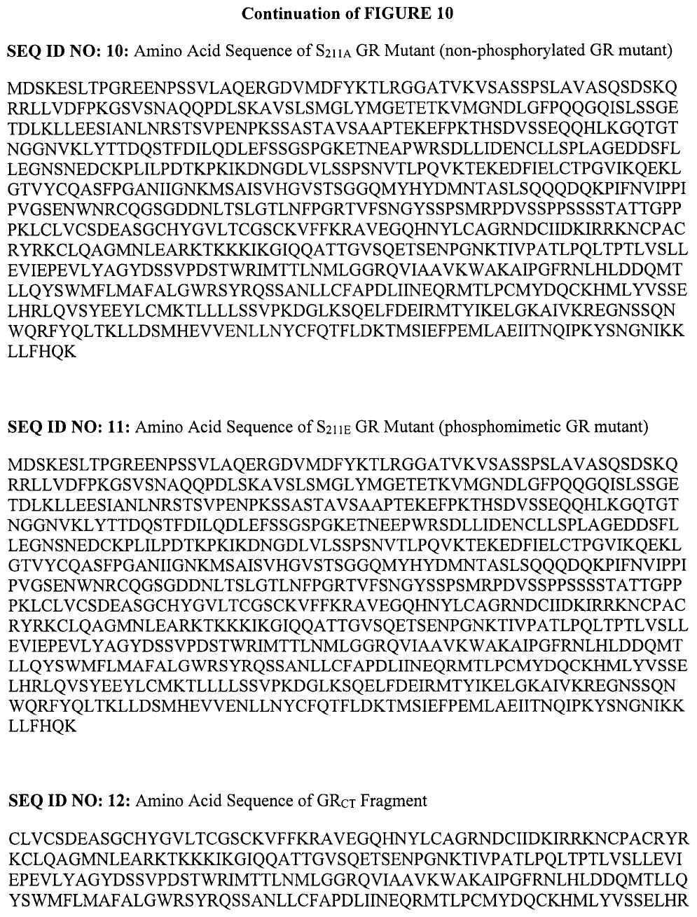

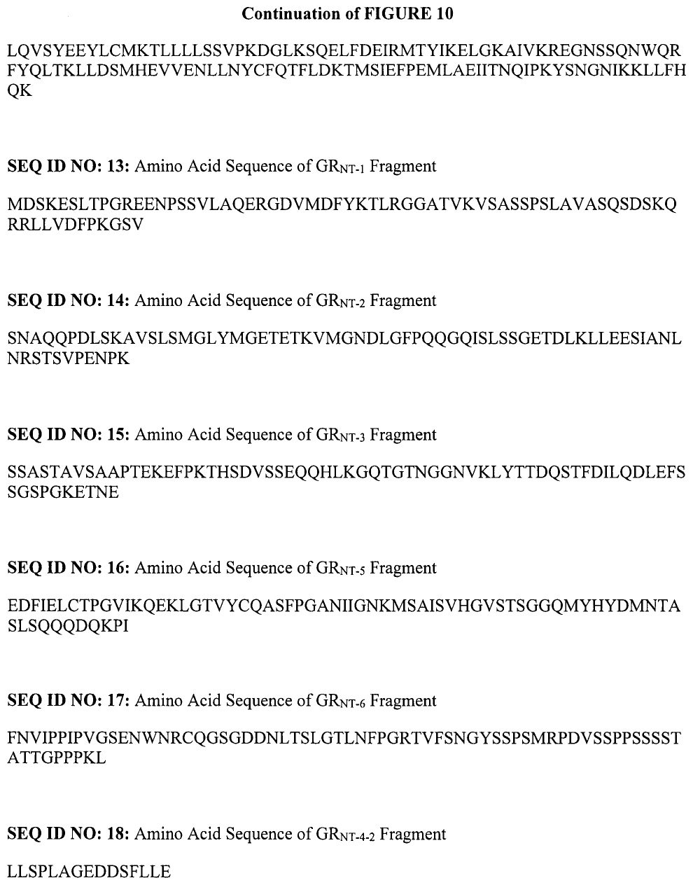

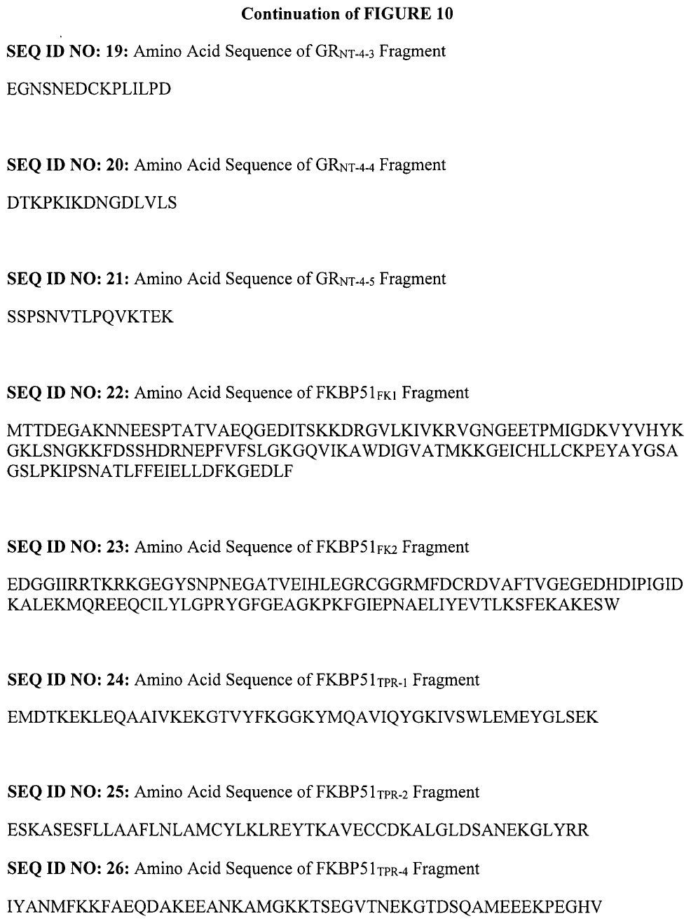

In certain embodiments, the agent may comprise, for example, a mimic of an N-terminal region of GR (GR_{NT}) which interacts with FKBP51 in the GR-FKBP51 complex. The amino acid sequence of human glucocorticoid receptor (GR) is shown in FIG. 10 as SEQ ID NO: 1, and the region defined as the N-terminal region of GR (GR_{NT}) is shown in FIG. 10 as SEQ ID NO: 2. In certain embodiments, the agent may comprise a mimic of a GR_{NT-4} region, or a GR_{NT-4-1} region, of GR. The region of GR defined as the GR_{NT-4} region is shown in FIG. 10 as SEQ ID NO: 3, and the region of GR defined as the GR_{NT-4-1} region is shown in FIG. 10 as SEQ ID NO: 4. In still further embodiments, the agent may comprise a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the full-length amino acid sequence S₂₁₁-L₂₂₅ of GR (also referred to herein as GR_{NT-4-1}, SEQ ID NO: 4), or any 7, 8, 9, 10, 11, 12, 13, or 14 consecutive amino acids thereof. In certain embodiments, the agent may comprise at least 7 amino acids, at least 8 amino acids, at least 9 amino acids, at least 10 amino acids, at least 11 amino acids, at least 12 amino acids, at least 13 amino acids, at least 14 amino acids, or at least 15 amino acids in length. In yet further embodiments of the agent, an amino acid residue of the polypeptide which corresponds to position S₂₁₁ of GR may comprise a wild-type (WT) residue, an S_{211A} mutant residue, or an S_{211E} mutant residue. In certain embodiments, the agent may comprise, for example, a polypeptide having the amino acid sequence S₂₁₁-L₂₂₅ of GR (see SEQ ID NO: 4 of FIG. 10), or a portion thereof or a functional mimic thereof (which may feature one or more conservative amino acid substitutions, for example) sufficiently long so as to bind FKBP51 and prevent complex formation. In certain embodiments, the agent may comprise at least 7 amino acids, at least 8 amino acids, at least 9 amino acids, at least 10 amino acids, at least 11 amino acids, at least 12 amino acids, at least 13 amino acids, at least 14 amino acids, or at least 15 amino acids in length.

As will be understood, the agents described above with reference to the GR sequence are designed according to the human GR sequence. For applications in non-human species, it is contemplated that sequences may, in certain embodiments, be adjusted based on amino acid sequence differences in GR between humans and the non-human species. That said, as described in detail in the Examples section below, the human GR-based agents described above were tested in a mouse model of PTSD, and a therapeutic benefit was still observed in these studies despite human and mice having slightly different GR sequences. For example, mouse GR is missing a cysteine residue toward the C-terminal of the relevant human S₂₁₁-L₂₂₅ region, instead having the sequence SPWRSDLLIDENLL. Nonetheless, an effect was observed despite the sequence variation.

In still further embodiments, the agent may comprise, for example, a mimic of a tetratricopeptide repeat (TPR) domain (FKBP51_{TPR}) of FKBP1 which interacts with GR in the GR-FKBP51 complex. The amino acid sequence of human FK506 Binding Protein 51 (FKBP51) is shown in FIG. 10 as SEQ ID NO: 5, and the region defined as the TPR domain (FKBP51_{TPR}) is shown in FIG. 10 as SEQ ID NO: 6. In yet another embodiment, the agent may comprise a mimic of an FKBP51_{TPR3} region of FKBP51. The region of FKBP51 defined as the FKBP51_{TPR3} region is shown in FIG. 10 as SEQ ID NO: 7. In still further embodiments, the agent may comprise a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the full-length amino acid sequence FKBP51_{TPR3} region of FKBP51, or any 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 consecutive amino acids thereof. In certain embodiments, the agent may comprise at least 7 amino acids, at least 8 amino acids, at least 9 amino acids, at least 10 amino acids, at least 11 amino acids, at least 12 amino acids, at least 13 amino acids, at least 14 amino acids, or at least 15 amino acids in length. In yet another embodiment, the agent may comprise, for example, a polypeptide having the amino acid sequence of the FKBP51_{TPR3} region (see SEQ ID NO: 7 of FIG. 10), or a portion thereof or a functional mimic thereof (which may feature one or more conservative amino acid substitutions, for example) sufficiently long so as to bind GR and prevent complex formation. In certain embodiments, the agent may comprise at least 7 amino acids, at least 8 amino acids, at least 9 amino acids, at least 10 amino acids, at least 11 amino acids, at least 12 amino acids, at least 13 amino acids, at least 14 amino acids, or at least 15 amino acids in length.

As will be understood, the agents described above with reference to the FKBP51 sequence are designed according to the human FKBP51 sequence. For applications in non-human species, it is contemplated that sequences may, in certain embodiments, be adjusted based on amino acid sequence differences in FKBP51 between humans and the non-human species. By way of example, FKBP51_{TPR3} domain of mouse FKBP51 differs from the human sequence, having the sequence GEAQLLMNDFESAKGDFEKVLAVNPQNRAARLQISMCQRKAKEHNERDRR, which is about 92% identical with the human sequence. That said, as described in detail in the Examples section below, human GR-based agents described herein were tested in a mouse model of PTSD, and a therapeutic benefit was still observed in these studies despite human and mice having different (albeit similar) FKBP51_{TPR3} domain sequences.

As will be understood, mimics as referred to herein, such as mimics of an N-terminal region of GR (GR_{NT}) which interacts with FKBP51 in the GR-FKBP51 complex, or mimics of a tetratricopeptide repeat (TPR) domain (FKBP51_{TPR}) of FKBP1 which interacts with GR in the GR-FKBP51 complex, may refer to any suitable mimic which presents binding features (i.e. electrostatic, steric, H-bonding, etc. . . . ) sufficiently close to the native polypeptide regions so as to be capable of binding GR, or FKBP51, in a manner which reduces levels of GR-FKBP51 complex in the subject. While peptide-based mimics are a preferred example used in the Examples section below, other mimics are also contemplated which need not be peptide-based, or which may be a combination of peptide and non-peptide moieties, for example. Where a peptide-based mimic is used, the peptide-based mimic may include amino acid sequence having at least some similarity (i.e. sequence identity) to the wild-type GR/FKBP51 region sequence, however peptides having low or no sequence identity to either wild-type region are also contemplated. For example, peptides having conservative amino acid substitutions, or peptides having no sequence similarity but featuring amino acid side chains still capable of binding the relevant regions of GR/FKBP51, are also contemplated.

In certain embodiments, the agent may be administered, or may be for administration, to the subject in generally any suitable manner such that at least a portion of the agent can access the brain of the subject. In certain embodiments, the agent may be administered to the subject in generally any suitable manner such that at least a portion of the agent can access the brain of the subject, which may in certain further embodiments include accessing the amygdala region and/or the prefrontal cortex region and/or the hippocampus region of the brain of the subject.

In certain embodiments, administration may be systemic administration, or may be local administration. For example, in certain embodiments, the administration may comprise direct injection to the brain, or to the amygdala, prefrontal cortex, and/or hippocampus region(s) thereof, for example. In certain embodiments, the administration may comprise a systemic administration. In certain embodiments, the administration may comprise, for example, intraperitoneal administration, intravenous administration, subcutaneous administration, oral administration, administration as a nasal spray, topical administration, or any suitable administration route suitable for the particular application and form of the agent being administered.

In certain embodiments, at least a portion of the agent may be administered to the amygdala and/or prefrontal cortex and/or hippocampus region(s) of the subject, either directly, or indirectly via systemic or local administration to the subject followed by diffusion or transport to the amygdala and/or prefrontal cortex and/or hippocampus region(s).

In certain embodiments, the agent may be formulated with (or may form a composition with) a delivery moiety, or delivery vehicle, which may facilitate translocation of the agent through a cell membrane and/or a blood brain barrier. Various delivery moieties (i.e. targeting peptides or other targeting functional groups, cell-penetrating peptides, antibodies, etc. . . . ) and delivery vehicles (i.e. liposomes, vesicles, nanoparticles, etc. . . . , including functionalized forms thereof) have been developed, including those designed for carrying cargo across the blood-brain barrier. By way of non-limiting example, certain delivery modalities, in particular cell-penetrating peptides, are reviewed in detail in Heitz et al., 2009 (British Journal of Pharmacology (2009), Twenty Years of Cell-Penetrating Peptides: From Molecular Mechanisms to Therapeutics; 157:195-206), which is herein incorporated by reference in its entirety. The person of skill in the art having regard to the teachings herein will be able to select a suitable delivery moiety or delivery vehicle to suit the particular application and agent being used.

In certain embodiments, for example, the delivery moiety may comprise a cell membrane transduction domain of a human immunodeficiency virus (HIV) transactivator of transcription (TAT) protein. By way of example, the delivery moiety may comprise a cell-membrane transduction domain of HIV-type 1 TAT protein. In certain embodiments, the delivery moiety may comprise an amino acid sequence of SEQ ID NO: 8 as shown in FIG. 10, or a sequence having at least about 80%, 85%, 90%, or 95% sequence identity therewith.

In certain embodiments, the agent may be bound (covalently, or non-covalently) with the delivery moiety or delivery vehicle, or may be encapsulated by the delivery moiety or delivery vehicle, for example. In certain embodiments, the agent may comprise a fusion protein, in which the agent is fused with a delivery moiety. In certain embodiments, for example, the agent may comprise a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence GRKKRRQRRRPQSPWRSDLLIDENCLL (SEQ ID NO: 9, as shown in FIG. 10), which is a fusion protein in which a portion of TAT is fused with a portion of GR. In certain embodiments, the agent may comprise a fusion polypeptide having the amino acid sequence GRKKRRQRRRPQSPWRSDLLIDENCLL (SEQ ID NO: 9, as shown in FIG. 10), also referred to herein as TAT-GRpep.

In still another embodiment, the methods or uses described herein may further comprise:
measuring a level of GR-FKBP51 complex in the subject, comparing the measured level to a reference or control level representative of a non-PTSD condition, and identifying the subject as a candidate for treatment where the measured level of GR-FKBP51 complex in the subject is elevated relative to the reference or control level.

In certain embodiments, administration of the agent may normalize decreased GR phosphorylation levels, may increase GR-FKBP52 complex levels, may increase nuclear translocation of GR, or any combination thereof, in the subject. In certain embodiments, administration of the agent may increase phosphorylation levels of position S₂₁₁ of GR in the subject.

In certain embodiments, for example, the agent may be administered to the subject having elevated levels of GR-FKBP51 complex following a traumatic event, in order to prevent or reduce subsequent emergence of PTSD.

In certain embodiments, for example, the agent may be administered to the subject at generally any time following a traumatic event. In certain embodiments, the agent may be administered so as to either reduce already increased GR-FKBP51 complex levels, or so as to prevent GR-FKBP51 complex levels from increasing.

In still further embodiments, administration of the agent may block emergence of PTSD fear-related behaviours, may inhibit the consolidation of cued fear memory, or both, in the aftermath of trauma.

In still further embodiments of the methods and uses described herein, the subject maybe a subject having a measured level of GR-FKBP51 complex which is elevated relative to a reference or control level representative of a non-PTSD condition or healthy condition.

In yet another embodiment, there is provided herein an agent which inhibits formation of a glucocorticoid receptor (GR)-FK506 Binding Protein 51 (FKBP51) complex (GR-FKBP51 complex), or which disrupts already formed GR-FKBP51 complex. In certain embodiments, the agent may comprise any suitable agent as described herein.

In certain embodiments, the agent may further comprise a delivery moiety or delivery vehicle as described herein, which may facilitate translocation of the agent through a cell membrane and/or a blood brain barrier.

In another embodiment, there is provided herein a polypeptide comprising a polypeptide sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence S₂₁₁-L₂₂₅ of GR (SEQ ID NO: 4, as shown in FIG. 10). In certain embodiments, an amino acid residue of the polypeptide which corresponds to position S₂₁₁ of GR may comprise a wild-type (WT) residue, an S_{211A} mutant residue, or an S_{211E} mutant residue. In certain embodiments, the polypeptide may comprise a polypeptide sequence having the amino acid sequence S₂₁₁-L₂₂₅ of GR (SEQ ID NO: 4, as shown in FIG. 10). As will be understood, in certain embodiments, the polypeptide may comprise an isolated polypeptide. In certain embodiments, the polypeptide may be part of a larger covalent, or non-covalent, structure. For example, the polypeptide may be part of a larger protein or polypeptide sequence or fusion protein, or may be covalently or non-covalently bound to other structures or moieties.

In certain embodiments, the polypeptide may further comprise a delivery moiety or delivery vehicle as described herein, which may facilitate translocation of the polypeptide through a cell membrane and/or a blood brain barrier.

In still another embodiment, there is provided herein a polypeptide comprising a polypeptide sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of the FKBP51_{TPR3} region (SEQ ID NO: 7, as shown in FIG. 10). As will be understood, in certain embodiments, the polypeptide may comprise an isolated polypeptide. In certain embodiments, the polypeptide may be part of a larger covalent, or non-covalent, structure. For example, the polypeptide may be part of a larger protein or polypeptide sequence or fusion protein, or may be covalently or non-covalently bound to other structures or moieties.

In certain embodiments, the polypeptide may further comprise a delivery moiety or delivery vehicle as described herein, which may facilitate translocation of the agent through a cell membrane and/or a blood brain barrier.

In yet another embodiment, there is provided herein a pharmaceutical composition comprising an agent as described herein, a polypeptide as described herein, or any combination thereof, and (optionally) a pharmaceutically acceptable excipient, diluent, or carrier.

Examples of suitable excipients, diluents, or carriers may include those described in, for example, Remington's Pharmaceutical Sciences or other such textbooks and references known to the person of skill in the art.

In certain embodiments, the pharmaceutical composition may further comprise a delivery moiety and/or a delivery vehicle as described herein. In certain embodiments, the delivery moiety or delivery vehicle may comprise a targeting moiety, a translocation moiety, a nanoparticle, or a nanovesicle configured for delivery of the pharmaceutical composition to the brain (or particular region(s) thereof, such as, for example, the amygdala and/or prefrontal cortex and/or hippocampus region(s)), for example).

Diagnosis of PTSD:

As described in detail herein, GR-FKBP51 complex levels may be indicative of PTSD (particularly in its sub-clinical, incipient stage). Accordingly, in certain embodiments, diagnostic methods based on GR-FKBP51 complex as a biomarker may provide, for example, in certain embodiments, a prophylactic tool, which may be, for example, for screening post-traumatic individuals for those who may be likely to develop PTSD before they show symptoms.

In another embodiment, there is provided herein a method for diagnosing a subject as having, or being at risk of developing, a post-traumatic stress disorder (PTSD), said method comprising:
measuring a level of a glucocorticoid receptor (GR)-FK506 Binding Protein 51 (FKBP51) complex (GR-FKBP51 complex) in the subject;
comparing the measured level to a reference or control level representative of a non-PTSD condition; and
identifying the subject as having, or being at risk of developing, PTSD where the measured level of GR-FKBP51 complex in the subject is elevated relative to the reference or control level.

In still another embodiment, there is provided herein a use of a glucocorticoid receptor (GR)-FK506 Binding Protein 51 (FKBP51) complex (GR-FKBP51 complex) for diagnosing a subject as having, or being at risk of developing, a post-traumatic stress disorder (PTSD), wherein a measured level of the GR-FKBP51 complex in the subject is indicative of the subject having, or being at risk of developing, PTSD when the measured level is elevated relative to a reference or control level representative of a non-PTSD condition.

In certain embodiments of the methods and uses described herein, the subject may be a subject who has experienced, or is at risk of experiencing, a severe psychological trauma.

As will be understood, in certain embodiments the reference or control level representative of a non-PTSD condition may comprise a reference or control level of GR-FKBP51 complex which is representative of levels found in a control population of healthy subjects, or a reference or control level of GR-FKBP51 complex which is representative of a baseline level of GR-FKBP51 complex in the subject prior to a trauma, for example. The person of skill in the art having regard to the teachings herein will be able to select a suitable reference or control level appropriate for the particular diagnostic application.

In certain embodiments, a subject may be identified as having, or being at risk of developing, PTSD where the measured level of GR-FKBP51 complex in the subject is elevated relative to the reference or control level by an increase of about 1.1 fold or greater; about 1.2 fold or greater; about 1.3 fold or greater; about 1.4 fold or greater; about 1.5 fold or greater; about 1.6 fold or greater; about 1.7 fold or greater; about 1.8 fold or greater; about 1.9 fold or greater; or about 2.0 fold or greater, for example. In certain embodiments, the increase may be about 1.40 fold or greater; about 1.41 folder or greater; about 1.42 fold or greater; about 1.43 fold or greater; about 1.44 fold or greater; about 1.45 fold or greater; about 1.46 fold or greater; about 1.47 fold or greater; about 1.48 fold or greater; about 1.49 fold or greater; about 1.50 fold or greater; about 1.51 fold or greater; about 1.52 fold or greater; about 1.53 fold or greater; about 1.54 fold or greater; about 1.55 fold or greater; about 1.56 fold or greater; about 1.57 fold or greater; about 1.58 fold or greater; about 1.59 fold or greater; or about 1.60 fold or greater, for example.

In certain embodiments, a subject may be identified as having, or being at risk of developing, PTSD where the measured level of GR-FKBP51 complex in the subject is elevated relative to the reference or control level by an increase of about 10% or greater; about 20% or greater; about 30% or greater; about 40% or greater; about 50% or greater; about 60% or greater; about 70% or greater; about 80% or greater; about 90% or greater; or about 100% or greater, for example. In certain embodiments, the increase may be about 40% or greater; about 41% or greater; about 42% or greater; about 43% or greater; about 44% or greater; about 45% or greater; about 46% or greater; about 47% or greater; about 48% or greater; about 49% or greater; about 50% or greater; about 51% or greater; about 52% or greater; about 53% or greater; about 54% or greater; about 55% or greater; about 56% or greater; about 57% or greater; about 58% or greater; about 59% or greater; or about 60% or greater, for example.

In certain embodiments, for example, the subject may be a subject having experienced a psychological trauma, and the subject may be subjected to diagnosis at generally any time following the traumatic event. In certain embodiments, the subject may be diagnosed within about 1 week, about 2 weeks, about 3 weeks, or about 4 weeks following the traumatic event. In certain embodiments, the subject may be diagnosed within about 1 month, about 2 months, about 3 months, about 4 months, about 5 months, about 6 months, about 7 months, about 8 months, about 9 months, about 10 months, about 11 months, or about 12 months following the traumatic event. In certain embodiments, the subject may be diagnosed within about 1 year, about 2 years, about 3 years, about 4 years, about 5 years, about 6 years, about 7 years, about 8 years, about 9 years, or about 10 years following the traumatic event. In certain embodiments, the subject may be diagnosed within about 1 decade, about 2 decades, about 3 decades, about 4 decades, or about 5 decades following the traumatic event.

In certain embodiments, the step of measuring may be performed using a sample of peripheral blood, or other blood sample, obtained from the subject. In certain embodiments, the sample may comprise lymphocytes isolated from a peripheral blood sample of the subject, for example.

As will be understood, in certain embodiments, the step of measuring may be performed using any suitable sample obtained from the subject which contains GR and FKBP51 protein components. In certain embodiments, the step of measuring may be performed using any suitable sample obtained from the subject which contains cells containing GR and FKBP51 protein components. In certain embodiments, the sample may comprise a sample of brain tissue, for example.

In certain embodiments, the step of measuring may be performed by enzyme-linked immunosorbent assay (ELISA). In certain embodiments, the ELISA may involve using an anti-FKBP51 antibody crosslinked or bound to a surface, and exposing/incubating the surface with a sample lysate. The surface may then be washed and blocked (using BSA, for example), followed by washing and then incubation with an anti-GR antibody and subsequent washing, followed by incubation with a secondary antibody and sequent washing, and then ECL activity may be measured, for example. As will be understood, various ELISA measurement techniques and/or assays may be designed for performing the step of measuring, and this example is not intended to be limiting. For example, an ELISA which uses anti-GR antibody followed by anti-FKBP51 antibody may be contemplated for the step of measuring. The skilled person having regard to the teachings herein will be aware of suitable ELISA, immunoassay, or other assay designs for use in the step of measuring.

In certain embodiments, the step of measuring may comprise co-immunoprecipitation and/or Western blotting to quantify the level of GR-FKBP51 complex. In certain embodiments, the step of measuring may comprise quantifying the level of GR-FKBP51 complex using ELISA.

In certain embodiments, when a pull-down or immunoprecipitation step is performed using an antibody specific for a first component of the GR-FKBP51 complex, and detection of a second component of the GR-FKBP51 complex is then performed using an antibody specific for said second component, then the antibody for the first component may be selected based on having good sensitivity, and the antibody for the second component may be selected based on having good target specificity, so as to provide good measurement of GR-FKBP51 complex levels.

In further embodiments, the method or use may further comprise measuring a level of GR S₂₁₁ phosphorylation in the subject, where a decreased level relative to a reference or control level representative of a non-PTSD condition is further indicative of the subject having, or being at risk of developing, PTSD.

In further embodiments, the method or use may further comprise measuring a level of GR in the nucleus of a neuron (or brain tissue) sample, or a lymphocyte sample (obtained from peripheral blood, for example), obtained from the subject, where a decreased level relative to a reference or control level representative of a non-PTSD condition is further indicative of the subject having, or being at risk of developing, PTSD.

In still further embodiments, the method or use may further comprise measuring a level of GR-FKBP52 complex in a sample (such as a brain tissue sample) obtained from the subject, where a decreased level relative to a reference or control level representative of a non-PTSD condition is further indicative of the subject having, or being at risk of developing, PTSD.

In yet another embodiment, the subject identified as having, or being at risk of developing, PTSD according to the methods herein may be further identified as a candidate for treatment by a treatment method as described herein. In certain embodiments the subject identified as having, or being at risk of developing, PTSD according to the methods herein may be subjected to PTSD treatment, either according to the treatment methods described herein, according to conventional PTSD treatment approaches, or both.

In certain embodiments, the diagnostic methods and uses described herein may further include a step of treating the subject with an agent which inhibits formation of a glucocorticoid receptor (GR)-FK506 Binding Protein 51 (FKBP51) complex (GR-FKBP51 complex), or which disrupts already formed GR-FKBP51 complex, as described above, and/or treating the subject with one or more standard PTSD therapies.

In yet another embodiment, there is provided herein a kit for diagnosing PTSD, said kit comprising at least one of:
an anti-glucocorticoid receptor (GR) antibody;
an anti-FK506 Binding Protein 51 (FKBP51) antibody;
an anti-GR-FKBP51 complex antibody; or
instructions for using the kit to perform a diagnostic method as described herein.

In certain embodiments, the kit may comprise an ELISA or immunoassay kit. In certain embodiments, the ELISA or immunoassay kit may comprise one or more of:
an anti-FKBP51 primary antibody;
an anti-GR primary antibody;
a secondary antibody specific for binding to one of the primary antibodies, wherein the secondary antibody may optionally be labelled, or conjugated with an enzyme for fluorescent or chemiluminescent detection;
an ECL (enhanced chemiluminescent) or other fluorescent or chemiluminescent substrate;
BSA or a blocking agent;
a wash buffer; and/or
instructions for using the kit to perform a diagnostic method as described herein.

In certain embodiments of the kits described herein, the anti-GR antibody, the anti-FKBP51 antibody, or both, may be immobilized or covalently bound to a surface or membrane.

Screening for PTSD Therapeutic Agents:

As described in detail herein, GR-FKBP51 complex, and inhibitors thereof which reduce levels of GR-FKBP51 complex in a PTSD subject, may provide for therapeutic options for PTSD treatment. Accordingly, also provided herein are methods for identifying PTSD therapeutic candidates for further investigation as PTSD therapeutic lead compounds.

By way of example, in an embodiment, there is provided herein a method for identifying PTSD therapeutic candidates, said method comprising:
exposing one or more candidate agents to a mixture of glucocorticoid receptor (GR) and FK506 Binding Protein 51 (FKBP51) under conditions in which GR and FKBP51 are able to form GR-FKBP51 complex; and
measuring a level of GR-FKBP51 complex formed in the presence of the one or more candidate agents;

wherein a reduction in the level of GR-FKBP51 complex formed in the presence of the one or more candidate agents, as compared to a corresponding reference level of GR-FKBP51 complex formed in the absence of the one or more candidate agents, identifies the one or more candidate agents as PTSD therapeutic candidates.

In certain embodiments, the one or more candidate agents may comprise, for example, a small-molecule library, such as those used in conventional high throughput screening for lead candidates in drug discovery.

In certain embodiments, drug candidate screening assays may comprise one or more of BRET, FRET, and/or SPR methods for drug candidate identification.

In certain embodiments, a BRET assay may be used in which one protein (i.e. one component of GR-FKBP51 complex—either GR, or FKBP51) is fused to (for example) a luciferase, and the other protein (i.e. the other binding partner of the GR-FKBP51 complex) may be bound to a fluorescent protein or fluorophore. Addition of substrate may lead to the emission of luciferase. If the two proteins interact, energy transfer may cause emission of the fluorescent protein. When a drug candidate is added which can block the interaction between GR and FKBP51, a decrease of BRET may be detected.

In another embodiment, a FRET assay may be used in which one protein (i.e. one component of GR-FKBP51 complex—either GR, or FKBP51) is fused to a first fluorescent protein or fluorophore, and the other protein (i.e. the other binding partner of the GR-FKBP51 complex) may be bound to a second fluorescent protein or fluorophore. The excitation spectrum of the second fluorescent protein or fluorophore may overlap with the emission spectrum of the first fluorescent protein or fluorophore. If the two proteins interact, then excitation of the first fluorescent protein or fluorophore may cause emission of the second fluorescent protein or fluorophore. When a drug candidate is added which can block the interaction between GR and FKBP51, a decrease of FRET may be detected.

In certain embodiments, an SPR assay may be used to detect binding of GR and FKBP51 (for example, FKBP51 TPR domain). By way of non-limiting example, HEK293 cells expressing FLAG-tagged GR bound onto a FLAG antibody-coated Dextran 50,000 DA chip may be used. In certain embodiments, a running buffer (such as PBS, pH 7.4, with 0.005% Tween 20) may be used with a flow rate of about 25 ul/min. With association time of, for example, about 300s and dissociation time of, for example, about 240s, binding signals may be determined by injecting various concentrations of the GST-FKBP51-TPR, for example. When a drug candidate is added which can block the interaction between GR and FKBP51, a decrease of SPR signal may be detected.

In certain embodiments of the above methods, when drug candidate is added which can block the interaction between GR and FKBP51, a decrease of BRET, FRET, or SPR signal would be detected, flagging the candidate as a PTSD therapeutic candidate.

As will be understood, the person of skill in the art having regard to the teachings herein will be aware of suitable BRET, FRET, SPR, and other assay designs suitable for drug candidate screening, and these examples are provided for illustrative and non-limiting purposes intended for the person of skill in the art.

### EXAMPLES—INVESTIGATION OF GR-FKBP1 COMPLEX, AND THERAPEUTIC AND DIAGNOSTIC APPLICATIONS THEREOF

In the following Examples, studies are described which identify that glucocorticoid receptor (GR) and FKBP51 (FK506 Binding Protein 51) form a protein complex that is elevated in fear-conditioned mice, an animal model for PTSD, and in PTSD patients compared to unaffected control subjects as well as subjects exposed to trauma without PTSD. Further, studies in which a peptide-based inhibitor was developed are described in which the inhibitor disrupted GR-FKBP51 binding, and in fear-conditioned mice, reduced freezing time. The peptide also normalized decreased GR phosphorylation, and increased both GR-FKBP52 binding and nuclear translocation of the GR in fear-conditioned mice. These findings may provide for methods of preventing or treating PTSD, methods for diagnosing PTSD, and/or agents, compositions, and/or kits therefor. A molecular mechanism contributing to PTSD is identified here in what is believed to be for the first time, and applied to the development of therapeutic and/or diagnostic approaches for PTSD.

Results

Higher FKBP51-GR Complex Levels in Peripheral Blood of PTSD Patients

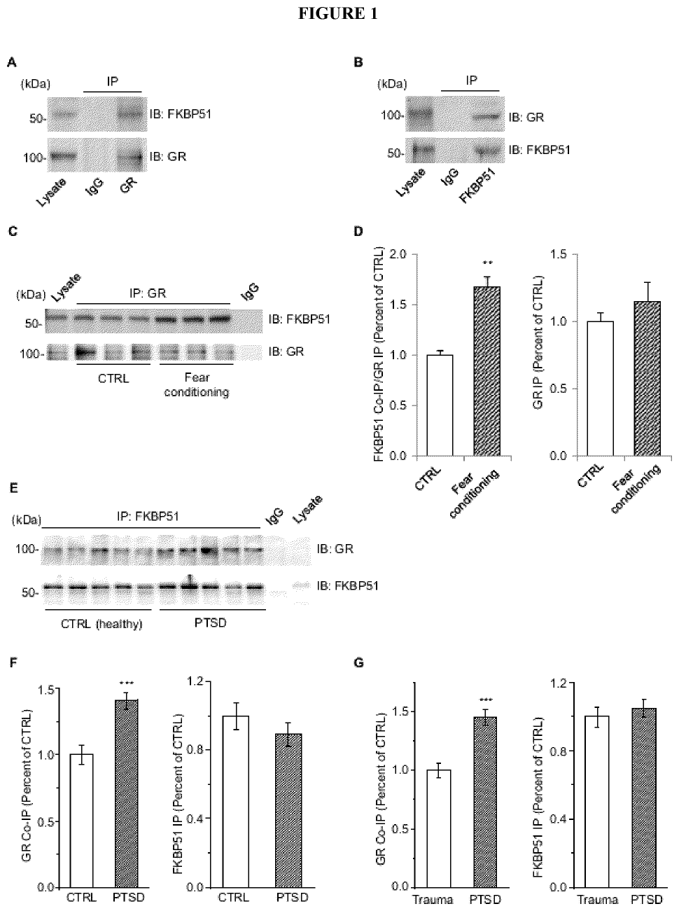

Studies were performed to investigate whether FKBP51 forms a protein complex with GR in mouse brain. As shown in FIG. 1A, a GR antibody, but not IgG, co-immunoprecipitated with FKBP51, while the FKBP51 antibody co-immunoprecipitates with GR (FIG. 1), suggesting the existence of a GR-FKBP51 complex. It was next examined whether the GR-FKBP51 complex is affected by fear-conditioned learning in the mouse, an animal model for dysregulated fear that occurs in PTSD.^{19, 20, 21} Cued fear conditioning consisted of five pairings of a light and tone (conditioned stimulus: CS) with a foot-shock (unconditioned stimulus: US). As shown in FIG. 1C-D, co-immunoprecipitation of FKBP51 by the GR antibody was significantly higher in fear conditioned mice vs. control mice (P<0.01, n=3, student's t-test), with no significant difference in direct immunoprecipitation of GR between the two groups.

Next, GR-FKBP51 complex levels were measured in the peripheral blood of PTSD patients recruited from Centre for Addiction and Mental Health (CAMH), University of Toronto. Equal amounts of protein from each sample were incubated with anti-FKBP51 antibody and the precipitated proteins were immunoblotted with either FKBP51 or GR antibody. Each Western blot included five samples per group and results were normalized against the mean of five control samples on the same blot. It was found that the GR-FKBP51 complex was significantly higher in peripheral blood from PTSD patients compared to healthy control subjects (n=22 each group; p<0.001, student's t-test; FIG. 1E-F). There was no significant difference in direct immunoprecipitation of FKBP51 between the two groups.

Evidence that the elevated GR-FKBP51 interaction is specific to PTSD and not just a biomarker of trauma exposure per se was next sought, by performing a replication and extension analysis in 42 subjects from the Grady Trauma Project in Atlanta, Ga.^{13, 22, 23} All subjects had significant trauma exposure, but while 21 had severe PTSD, the age, sex and race-matched trauma controls had few symptoms. Significantly elevated GR-FKBP51 was found in peripheral blood from PTSD subjects vs trauma controls (p<0.001, student's t-test, FIG. 1G), with no significant difference in direct immunoprecipitation of FKBP51 between the two groups. These combined data indicates that the FKBP51-GR complex may be a biomarker for PTSD that may facilitate early detection of those at higher risk for PTSD after a traumatic event.

GR Interacts with FKBP51 Through the GR_{NT-4-1} (S₂₁₁-L₂₂₅) Region

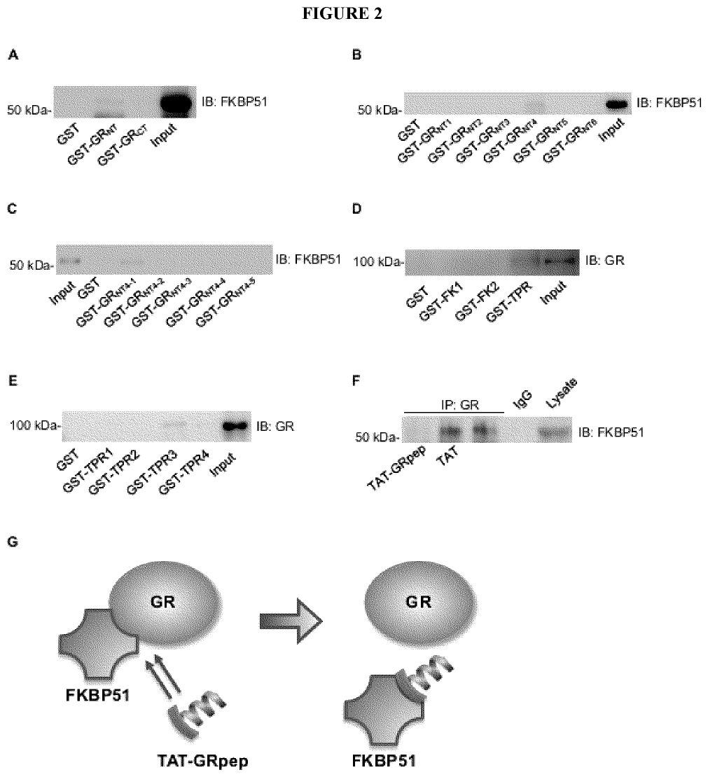

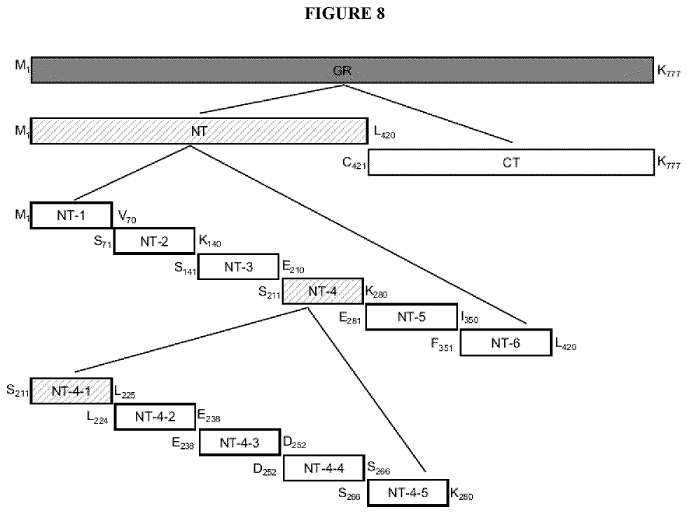

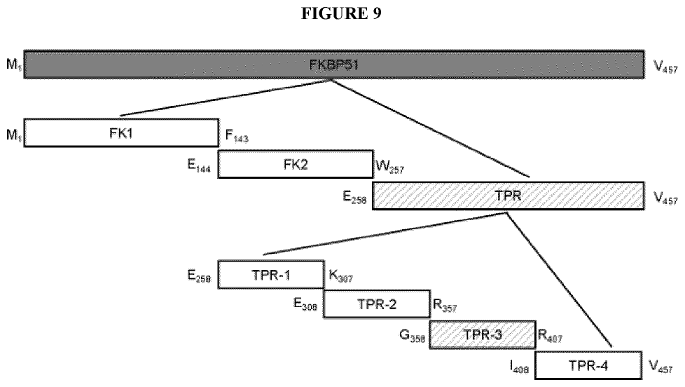

The present inventors identified that if increased GR-FKBP51 complex levels are part of the pathophysiology of PTSD, then disrupting the complex may rescue behaviors related to PTSD. To develop an agent (in this example, a peptide-based agent) capable of blocking the GR-FKBP51 interaction, experiments were first performed to identify the regions of the GR that are important to bind FKBP51 (and vice versa). Initially, GST-fusion proteins were used encoding the amino-terminus (NT) of GR (GR_{NT}) and the carboxyl-terminus (CT) of GR (GR_{CT}) for affinity purification. As shown in FIG. 2A, GST-GR_{NT}, but not GST-GR_{CT}, precipitated FKBP51, suggesting that GR_{NT} was sufficient for the FKBP51-GR interaction. Extending this strategy, GR_{NT} fragments were examined (see FIG. 8, where the 6 tested GR_{NT} fragments, NT-1, NT-2, NT-3, NT-4, NT-5, and NT-6 are shown). Of these, the NT-4 fragment showed the best precipitation of FKBP51, and so this fragment was further fragmented into an additional 5 fragments (NT-4-1, NT-4-2, NT-4-3, NT-4-4, and NT-4-5; see FIG. 8) which were tested, and it was concluded that GR_{NT-4-1} is (or contains) the region interacting with FKBP51 (FIG. 2B-C). Similarly, it was determined that the TPR (tetratricopeptide repeat) domains of FKBP51 bind GR (FIG. 2D). In these studies, GST-fusion proteins of 3 different portions of the FKBP51 protein were prepared (FK1, FK2, and TPR, see FIG. 9) and tested. The TPR fragment was identified as containing the relevant interaction region. Further dissecting the FKBP51_{TPR} into smaller fragments (see FIG. 9, depicting the 4 TPR fragments tested, labelled as TPR-1, TPR-2, TPR-3, and TPR-4) revealed that FKBP51_{TPR3} is (or contains) the region interacting with GR (FIG. 2E).

Sequences of GR and FKBP51, as well as the various fragments thereof used in the above testing, are shown in the following Table, and in FIG. 10.

TABLE 1
GR and FKBP51 fragments used to
identify the interaction region
of GR-FKBP51 complex.
GR and FKBP51 fragments used to identify
the interaction region of GR-FKBP51
complex

| | Amino Acid Sequence |
|---|---|
| Label | (not including GST tag) |
| GR | MDSKESLTPGREENPSSVLA |
| (SEQ ID | QERGDVMDFYKTLRGGATVK |
| NO: 1) | VSASSPSLAVASQSDSKQRR |
| | LLVDFPKGSVSNAQQPDLSK |
| | AVSLSMGLYMGETETKVMGN |
| | DLGFPQQGQISLSSGETDLK |
| | LLEESIANLNRSTSVPENPK |
| | SSASTAVSAAPTEKEFPKTH |
| | SDVSSEQQHLKGQTGTNGGN |
| | VKLYTTDQSTFDILQDLEFS |
| | SGSPGKETNESPWRSDLLID |
| | ENCLLSPLAGEDDSFLLEGN |
| | SNEDCKPLILPDTKPKIKDN |
| | GDLVLSSPSNVTLPQVKTEK |
| | EDFIELCTPGVIKQEKLGTV |
| | YCQASFPGANIIGNKMSAIS |
| | VHGVSTSGGQMYHYDMNTAS |
| | LSQQQDQKPIFNVIPPIPVG |
| | SENWNRCQGSGDDNLTSLGT |
| | LNFPGRTVFSNGYSSPSMRP |
| | DVSSPPSSSSTATTGPPPKL |
| | CLVCSDEASGCHYGVLTCGS |
| | CKVFFKRAVEGQHNYLCAGR |
| | NDCIIDKIRRKNCPACRYRK |
| | CLQAGMNLEARKTKKKIKGI |
| | QQATTGVSQETSENPGNKTI |
| | VPATLPQLTPTLVSLLEVIE |
| | PEVLYAGYDSSVPDSTWRIM |
| | TTLNMLGGRQVIAAVKWAKA |
| | IPGFRNLHLDDQMTLLQYSW |
| | MFLMAFALGWRSYRQSSANL |
| | LCFAPDLIINEQRMTLPCMY |
| | DQCKHMLYVSSELHRLQVSY |
| | EEYLCMKTLLLLSSVPKDGL |
| | KSQELFDEIRMTYIKELGKA |
| | IVKREGNSSQNWQRFYQLTK |
| | LLDSMHEVVENLLNYCFQTF |
| | LDKTMSIEFPEMLAEIITNQ |
| | IPKYSNGNIKKLLFHQK |
| GR_{NT} | MDSKESLTPGREENPSSVLA |
| (SEQ ID | QERGDVMDFYKTLRGGATVK |
| NO: 2) | VSASSPSLAVASQSDSKQRR |
| | LLVDFPKGSVSNAQQPDLSK |
| | AVSLSMGLYMGETETKVMGN |
| | DLGFPQQGQISLSSGETDLK |
| | LLEESIANLNRSTSVPENPK |
| | SSASTAVSAAPTEKEFPKTH |
| | SDVSSEQQHLKGQTGTNGGN |
| | VKLYTTDQSTFDILQDLEFS |
| | SGSPGKETNESPWRSDLLID |
| | ENCLLSPLAGEDDSFLLEGN |
| | SNEDCKPLILPDTKPKIKDN |
| | GDLVLSSPSNVTLPQVKTEK |
| | ED |
| | FIELCTPGVIKQEKLGTVYC |
| | QASFPGANIIGNKMSAISVH |
| | GVSTSGGQMYHYDMNTASLS |
| | QQQDQKPIFNVTPPIPVGSE |
| | NWNRCQGSGDDNLTSLGTLN |
| | FPGRTVFSNGYSSPSMRPDV |
| | SSPPSSSSTATTGPPPKL |
| GR_{CT} | CLVCSDEASGCHYGVLTCGS |
| (SEQ ID | CKVFFKRAVEGQHNYLCAGR |
| NO: 12) | NDCIIDKIRRKNCPACRYRK |
| | CLQAGMNLEARKTKKKIKGI |
| | QQATTGVSQETSENPGNKTI |
| | VPATLPQLTPTLVSLLEVIE |
| | PEVLYAGYDSSVPDSTWRIM |
| | TTLNMLGGRQVIAAVKWAKA |
| | IPGFRNLHLDDQMTLLQYSW |
| | MFLMAFALGWRSYRQSSANL |
| | LCFAPDLIINEQRMTLPCMY |
| | DQCKHMLYVSSELHRLQVSY |
| | EEYLCMKTLLLLSSVPKDGL |
| | KSQELFDEIRMTYIKELGKA |
| | IVKREGNSSQNWQRFYQLTK |
| | LLDSMHEVVENLLNYCFQTF |
| | LDKTMSIEFPEMLAEIITNQ |
| | IPKYSNGNIKKLLFHQK |
| GR_{NT-1} | MDSKESLTPGREENPSSVLA |
| (SEQ ID | QERGDVMDFYKTLRGGATVK |
| NO: 13) | VSASSPSLAVASQSDSKQRR |
| | LLVDFPKGSV |
| GR_{NT-2} | SNAQQPDLSKAVSLSMGLYM |
| (SEQ ID | GETETKVMGNDLGFPQQGQI |
| NO: 14) | SLSSGETDLKLLEESIANLN |
| | RSTSVPENPK |
| GR_{NT-3} | SSASTAVSAAPTEKEFPKTH |
| (SEQ ID | SDVSSEQQHLKGQTGTNGGN |
| NO: 15) | VKLYTTDQSTFDILQDLEFS |
| | SGSPGKETNE |
| GR_{NT-4} | SPWRSDLLIDENCLLSPLAG |
| (SEQ ID | EDDSFLLEGNSNEDCKPLIL |
| NO: 3) | PDTKPKIKDNGDLVLSSPSN |
| | VTLPQVKTEK |
| GR_{NT-5} | EDFIELCTPGVIKQEKLGTV |
| (SEQ ID | YCQASFPGANIIGNKMSAIS |
| NO: 16) | VHGVSTSGGQMYHYDMNTAS |
| | LSQQQDQKPI |
| GR_{NT-6} | FNVIPPIPVGSENWNRCQGS |
| (SEQ ID | GDDNLTSLGTLNFPGRTVFS |
| NO: 17) | NGYSSPSMRPDVSSPPSSSS |
| | TATTGPPPKL |
| GR_{NT-4-1} | SPWRSDLLIDENCLL |
| (SEQ ID | |
| NO: 4) | |
| GR_{NT-4-2} | LLSPLAGEDDSFLLE |
| (SEQ ID | |
| NO: 18) | |
| GR_{NT-4-3} | EGNSNEDCKPLILPD |
| (SEQ ID | |
| NO: 19) | |
| GR_{NT-4-4} | DTKPKIKDNGDLVLS |
| (SEQ ID | |
| NO: 20) | |
| GR_{NT-4-5} | SSPSNVTLPQVKTEK |
| (SEQ ID | |
| NO: 21) | |
| FKBP51 | MTTDEGAKNNEESPTATVAE |
| (SEQ ID | QGEDITSKKDRGVLKIVKRV |
| NO: 5) | GNGEETPMIGDKVYVHYKGK |
| | LSNGKKFDSSHDRNEPFVFS |
| | LGKGQVIKAWDIGVATMKKG |
| | EICHLLCKPEYAYGSAGSLP |
| | KIPSNATLFFEIELLDFKGE |
| | DLFEDGGIIRRTKRKGEGYS |
| | NPNEGATVEIHLEGRCGGRM |
| | FDCRDVAFTVGEGEDHDIPI |
| | GIDKALEKMQREEQCILYLG |
| | PRYGFGEAGKPKFGIEPNAE |
| | LIYEVTLKSFEKAKESWEMD |
| | TKEKLEQAAIVKEKGTVYFK |
| | GGKYMQAVIQYGKIVSWLEM |
| | EYGLSEKESKASESFLLAAF |
| | LNLAMCYLKLREYTKAVECC |
| | DKALGLDSANEKGLYRRGEA |
| | QLLMNEFESAKGDFEKVLEV |
| | NPQNKAARLQISMCQKKAKE |
| | HNERDRRIYANMFKKFAEQD |
| | AKEEANKAMGKKTSEGVTNE |
| | KGTDSQAMEEEKPEGHV |
| FKBP51FK1 | MTTDEGAKNNEESPTATVAE |
| (SEQ ID | QGEDITSKKDRGVLKIVKRV |
| NO: 22) | GNGEETPMIGDKVYVHYKGK |
| | LSNGKKFDSSHDRNEPFVFS |
| | LGKGQVTKAWDIGVATMKKG |
| | EICFILLCKPEYAYGSAGSL |
| | PKIPSNATLFFEIELLDFKG |
| | EDLF |
| FKBP51FK2 | EDGGIIRRTKRKGEGYSNPN |
| (SEQ ID | EGATVEIHLEGRCGGRMFDC |
| NO: 23) | RDVAFTVGEGEDHDIPIGID |
| | KALEKMQREEQCILYLGPRY |
| | GFGEAGKPKFGIEPNAELIY |
| | EVTLKSFEKAKESW |
| FKBP51TPR | EMDTKEKLEQAAIVKEKGTV |
| (SEQ ID | YFKGGKYMQAVIQYGKIVSW |
| NO: 6) | LEMEYGLSEKESKASESFLL |
| | AAFLNLAMCYLKLREYTKAV |
| | ECCDKALGLDSANEKGLYRR |
| | GEAQLLMNEFESAKGDFEKV |
| | LEVNPQNKAARLQISMCQKK |
| | AKEHNERDRRIYANMFKKFA |
| | EQDAKEEANKAMGKKTSEGV |
| | TNEKGTDSQAMEEEKPEGHV |
| FKBP51_{TPR-1} | EMDTKEKLEQAAIVKEKGTV |
| (SEQ ID | YFKGGKYMQAVIQYGKIVSW |
| NO: 24) | LEMEYGLSEK |
| FKBP51_{TPR-2} | ESKASESFLLAAFLNLAMCY |
| (SEQ ID | LKLREYTKAVECCDKALGLD |
| NO: 25) | SANEKGLYRR |
| FKBP51_{TPR-3} | GEAQLLMNEFESAKGDFEKV |
| (SEQ ID | LEVNPQNKAARLQISMCQKK |
| NO: 7) | AKEHNERDRR |
| FKBP51_{TPR-4} | IYANMFKKFAEQDAKEEANK |
| (SEQ ID | AMGKKTSEGVTNEKGTDSQA |
| NO: 26) | MEEEKPEGHV |

A peptide emulating the amino acid sequence of GR_{NT-4-1} was synthesized (GRpep [S₂₁₁-L₂₂₅]), and fused to the cell-membrane transduction domain of the human immunodeficiency virus-type 1 TAT protein (see SEQ ID NO: 8 of FIG. 10) as previously described.^{24, 25, 26} If the GR_{NT-4-1} region is important for GR-FKBP51 binding, then GRpep should disrupt the GR-FKBP51 complex by competing with GR for FKBP51. Results indicate that TAT-GRpep (see SEQ ID NO: 9, shown in FIG. 10), but not the control TAT peptide, reduced GR-FKBP51 levels in mouse brain (FIG. 2F-G). Thus, it is concluded that the GR_{NT-4-1} region is important for GR-FKBP51 complex formation, and that GRpep disrupted the GR-FKBP51 complex.

Direct Injection of TAT-GRpep into Amygdala Reduces Freezing

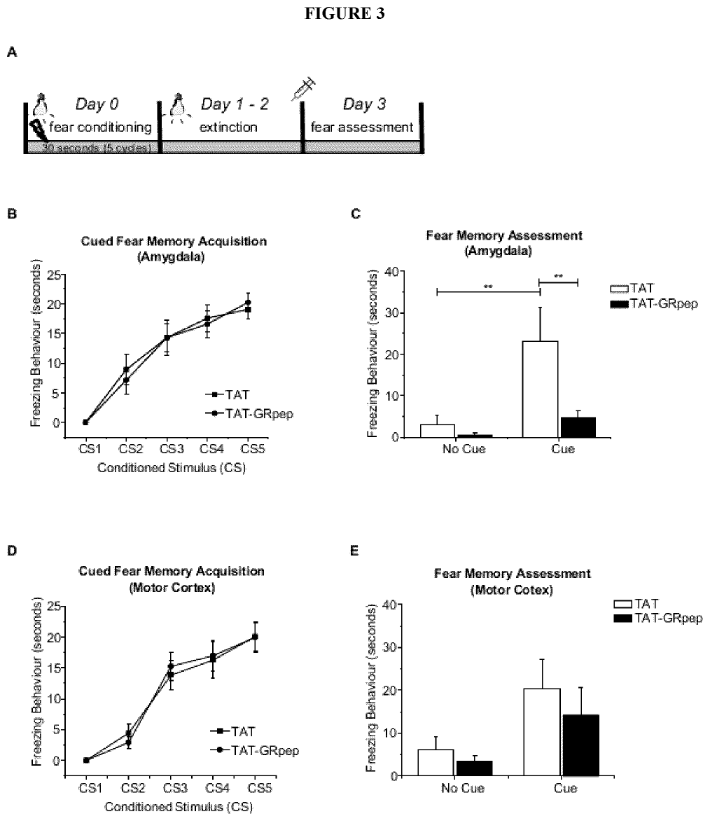

Next, testing was performed to determine whether disrupting the GR-FKBP51 complex could reduce freezing behaviors in fear-conditioned mice as a model for PTSD-related symptoms. As illustrated in FIG. 3A, two randomized groups of animals underwent five rounds of CS-US pairings (CS=light, US=0.5 mA foot shock for 1 second). The animals were then subjected to two days of extinction, where the same cues were presented without foot shock, before cued fear memory was assessed. One hour before the fear memory assessment, animals were injected with either control peptide (TAT) or treatment peptide (TAT-GRpep) into the right amygdala. The amygdala was targeted because it is critical for fear and emotional learning.²⁷ As shown in FIG. 3B, the two groups did not differ in the acquisition of cued-fear memory (two-way ANOVA with repeated measures, F_{1,21}=0.25, p=NS). However, two-way ANOVA confirmed a significant effect of the cue (F1, 46=10.387, p<0.01) and peptide (F_{1,46}=7.708, p<0.01). These results show that TAT-GRpep attenuated cued fear memory expression since these animals had significantly less freezing than controls (p<0.01; FIG. 3C). To demonstrate the anatomical specificity of TAT-GRpep effects, the same experiments were repeated, but infused peptide into the motor cortex instead of the amygdala. As shown in FIG. 3D, both groups acquired the memory of cued-fear (two-way ANOVA with repeated measures, F_{1,20}=0.03, p=NS). However, injecting TAT-GRpep into the motor cortex did not affect freezing (F_{1,20}=0.353, p=NS), nor was there an interaction effect (F_{1,20}=0.293, p=NS). Both treatment groups reacted similarly to the cue (p=NS; FIG. 3E). These data show that TAT-GRpep, when delivered to the amygdala, blocks the emergence of PTSD-like fear behaviours in the mouse, supporting a potential therapeutic approach for PTSD.

Systemic TAT-GRpep Delivery Reduces Freezing

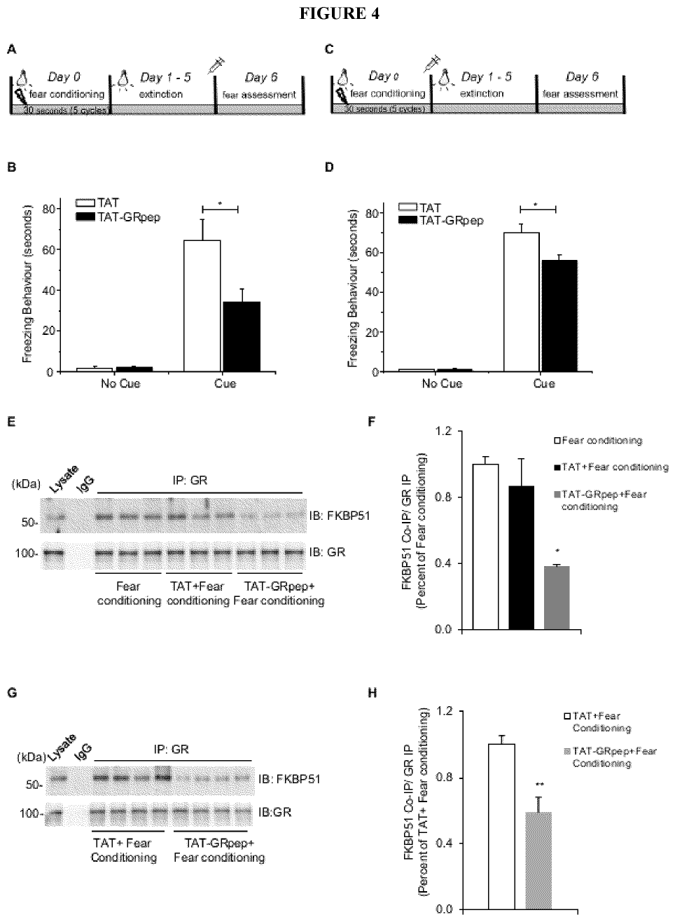

Next, testing was performed to determine whether TAT-GRpep can block the emergence of PTSD-like fear-related behaviours when administrated systemically. Animals underwent five rounds of CS-US pairings followed by 5 days of extinction training before the expression of fear in response to the learned cue was assessed (FIG. 4A). Mice were injected with either TAT-GRpep or TAT intraperitoneally (3 nmol/g, i.p.) one hour before the test. As shown in FIG. 4B, there was no difference in freezing behavior between the TAT-GRpep and control groups during the habituation period without CS (p=NS). However, with the CS, TAT-GRpep significantly reduced freezing compared to the TAT peptide (34.1±6.7 vs. 64.2±10.7 seconds, two-way ANOVA with repeated measures, p<0.05), confirming that TAT-GRpep may block the emergence of PTSD-like fear-related behaviour(s).

Furthermore, testing was performed to determine whether disrupting the GR-FKBP51 complex reduced consolidation of cued fear memory. Thus, TAT or TAT-GRpep was administered two hours after the end of fear conditioning training (FIG. 4C), before five consecutive daily extinction sessions of cue exposure without shock. 24 hours after the last extinction session, neither treatment group froze without the CS, but with the CS, TAT-GRpep treatment significantly reduced freezing compared to the TAT peptide (55.9±3.3 vs. 71.0±3.6 seconds, two-way ANOVA with repeated measures, p<0.05, FIG. 4D). The ability of TAT-GRpep to disrupt the GR-FKBP51 complex in fear-conditioned mice was confirmed with co-immunoprecipitation in both brain tissues (FIGS. 4E-F) and peripheral blood samples (FIG. 4G-H). These results suggest that disruption of the GR-FKBP51 complex not only blocked the emergence of PTSD-like fear-related behaviours, but inhibited the consolidation of cued fear memory. This may therefore indicate a potential approach to both preventing and treating PTSD-like behaviour in the aftermath of trauma, for example.

GR-FKBP51 Complex Formation Reduces GR Ser₂₁₁ (S₂₁₁) Phosphorylation

Next, how the GR-FKBP51 complex regulates GR signaling was investigated by studying whether the GR-FKBP51 complex affects GR regulatory mechanisms such as phosphorylation, and how GR-FKBP51 interactions affect GR nuclear translocation, the canonical measure of changes in GR function.²⁸ Activation of GR induces phosphorylation of specific GR sites in addition to basal GR phosphorylation.²⁹ In general, phosphorylation of GR protects it from degradation.^{30,31} S₂₁₁ is a known GR phosphorylation site, and is located within the GR-FKBP51 interacting region, and S₂₁₁ phosphorylation increases recruitment of GR to target genes.³²

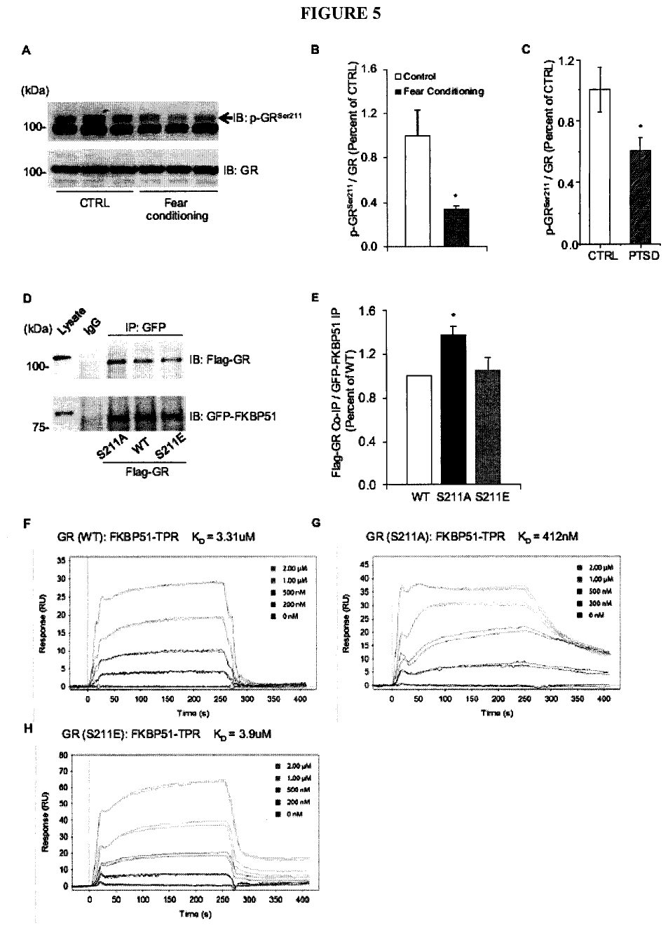

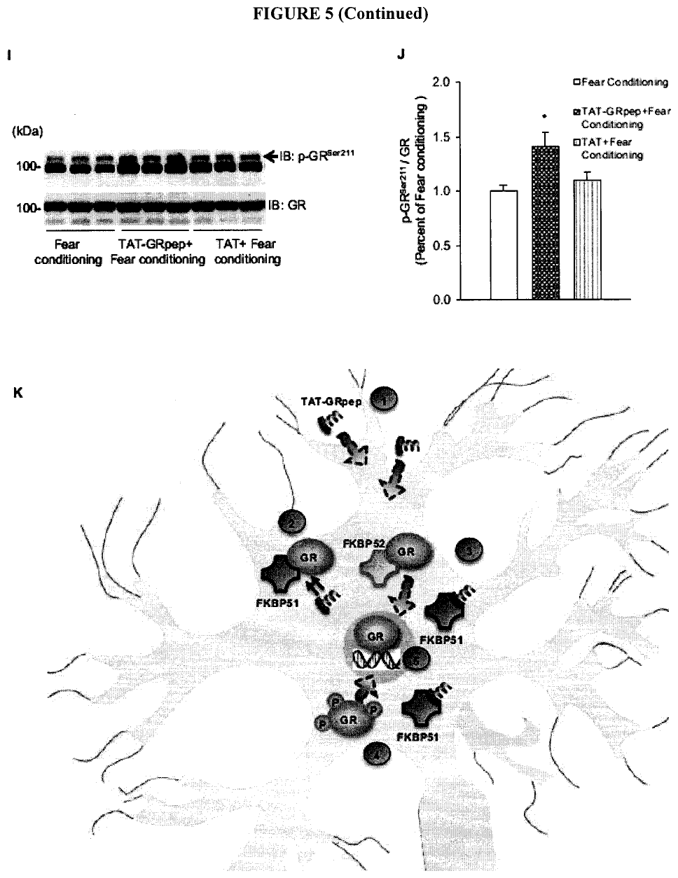

Thus, it was investigated whether the increased GR-FKBP51 seen in fear-conditioned mice and PTSD patients was associated with altered GR S₂₁₁ phosphorylation. As shown in FIG. 5A-B, S₂₁₁ phosphorylation was significantly decreased in fear-conditioned mice compared to control mice (n=3, p<0.05, t-test). Similar results were also seen in samples from PTSD patients (FIG. 5C n=12, p<0.05, t-test). To probe further into how S₂₁₁ phosphorylation and GR-FKBP51 are related, two GR mutants were generated (see SEQ ID NOs: 10 (S_{211A}) and 11 (S_{211E}) of FIG. 10). S_{211A} is a non-phosphorylated GR mutant and S_{211E} is phosphomimetic GR mutant. S_{211A} GR-FKBP51 complex levels were significantly higher than wild-type (WT) GR-FKBP51 or S_{211E} GR-FKBP51 levels (FIG. 5D-E, n=3, p<0.05, one-way ANOVA), suggesting that GR-FKBP51 complex formation may impede S₂₁₁ phosphorylation.

To further investigate these findings, surface plasmon resonance (SPR) was used to estimate association and dissociation rates between GR and FKBP51. As shown in FIG. 5F-H, the non-phosphorylated S_{211A} GR mutant has a much higher affinity for FKBP51 than the wild-type (WT) GR as well as the phosphomimetic S_{211E} GR mutant (S_{211A} GR K_{D}=412±7 nM; WT GR K_{D}=3.31±0.04 μM; S_{211E} GR KD=3.90±0.01 μM). Thus, GR S₂₁₁ phosphorylation seems to reduce GR affinity for FKBP51. Conversely, disruption of the GR-FKBP51 complex with TAT-GRpep could increase GR S₂₁₁ phosphorylation, which is what was observed when comparing fear-conditioned mice treated with TAT-GRpep vs. TAT control or no treatment (FIG. 5 I-J).

TAT-GRpep Facilitates GR-FKBP52 Complex Formation and GR Nuclear Translocation

Previous studies have suggested that impaired GR S₂₁₁ phosphorylation may decrease GR nuclear translocation.³³ Others have suggested that increasing levels of free GR may promote GR-FKBP52 (aka FKBP4) complex formation, leading to GR nuclear translocation, where GR may activate or repress transcription of target genes.³⁴

In the present experiments, it was observed that TAT-GRpep blocked the decrease in GR S₂₁₁ phosphorylation observed in the fear-conditioned animals. Thus, without wishing to be bound by theory, it is hypothesized herein that the reduction of PTSD-like behaviors by TAT-GRpep may occur in the following sequence: dissociation of GR-FKBP51 leads to more GR-FKBP52 complex and greater GR S₂₁₁ phosphorylation, both of which may facilitate GR nuclear translocation (see working model FIG. 5K).

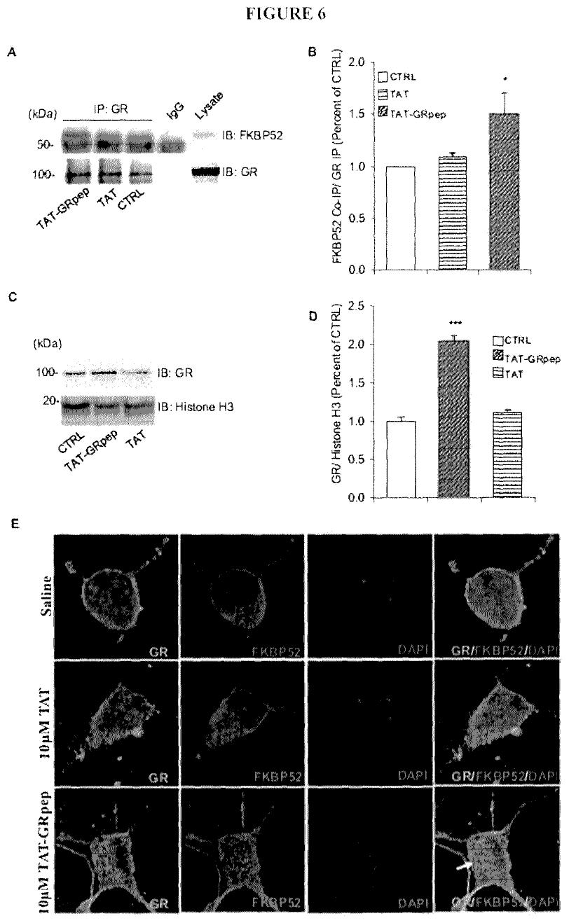

To test this hypothesis, the effect of TAT-GRpep on GR-FKBP52 levels was examined using co-immunoprecipitation. As shown in FIG. 6A-B, TAT-GRpep treatment significantly increased the amount of GR-FKBP52 complex (n=3, p<0.05, one-way ANOVA) and the proportion of GR in the nucleus (FIG. 6C-D n=3, p<0.001, one-way ANOVA). Similar results were also obtained using confocal microscope in primary mouse neuron cultures. As shown in FIG. 6E, GR and FKBP52 co-localized more with TAT-GRpep treatment vs. with saline or control peptide treatment. In addition, TAT-GRpep markedly increased nuclear GR levels, suggesting that blocking the GR-FKBP51 interaction may affect both GR-FKBP52 complex formation and nuclear translocation.

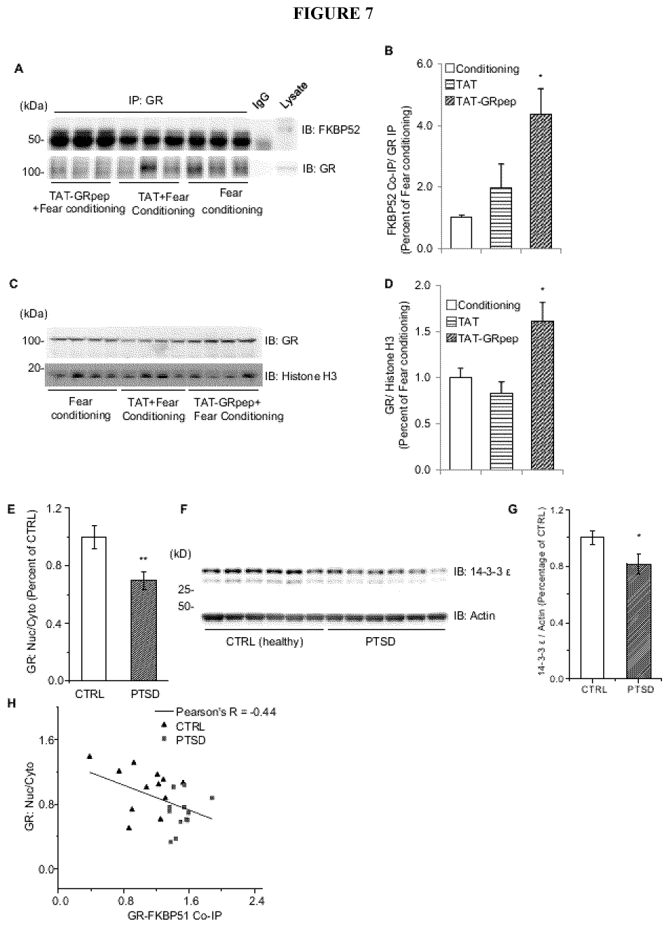

Based on these results, it was contemplated that TAT-GRpep may also increase GR-FKBP52 complex formation and GR nuclear expression when administered to fear conditioned mice. Indeed, significant increases in GR-FKBP52 complex levels were found in fear-conditioned mice after TAT-GRpep treatment (FIG. 7A-B p<0.05, n=3, one-way ANOVA). GR nuclear localization was also increased by TAT-GRpep in fear-conditioned mice (FIG. 7C-D p<0.05, n=4, one-way ANOVA), which is consistent with data showing that the Toronto patients with PTSD have a lower proportion of GR in the nucleus compared to healthy control subjects (FIG. 7E, p<0.01, n=12). Interestingly, the decreased expression of GR in the nucleus was also reflected functionally by the lower GR transcription activity as the expression of 14-3-3 ε, a direct target protein of GR,^{35, 36} was decreased in the same group of patients (FIG. 7F-G, p<0.05). Further analysis suggested that higher GR-FKBP51 complex levels are correlated with a lower proportion of the GR in the nucleus (Pearson's r=−0.44; p=0.030) (FIG. 7F). Without wishing to be bound by theory, these data support a hypothesis that an elevated GR-FKBP51 complex may lead to less GR nuclear translocation, which may result in decreased GR modulation of transcriptional activity.

### DISCUSSION

Direct evidence is provided for a GR-FKBP51 protein complex that is elevated in peripheral blood samples from PTSD patients and in fear-conditioned mice. A blocking peptide, TAT-GRpep, was synthesized that contains the same amino acid sequence as the region on GR that binds FKBP51, and this peptide blocks both the acquisition and recall of fear memories in mice. To investigate potential molecular mechanisms, phosphorylation of GR at S₂₁₁ was examined, which is within the region that interacts with FKBP51, and it was found that S₂₁₁ phosphorylation was decreased in both PTSD patients and fear-conditioned mice. To determine whether S₂₁₁ phosphorylation may affect GR-FKBP51 binding, two S₂₁₁ mutant GR proteins that mimic the phosphorylated and non-phosphorylated forms of GR at S₂₁₁ were used. It was found that the non-phosphorylated S₂₁₁ mutant form of GR had higher binding to FKBP51, and SPR confirmed a much lower K_{D} for the non-phosphorylated S₂₁₁ mutant form of GR. TAT-GRpep enhanced phosphorylation of GR at S₂₁₁ and also promoted binding of GR to FKBP52 and nuclear translocation of GR. Both PTSD patients and fear conditioned mice had relatively low GR in the nucleus, and TAT-GRpep increased GR nuclear localization in fear-conditioned mice. In PTSD patients, the lower GR in nucleus results in lower GR transcriptional modulation as demonstrated by the decreased expression of 14-3-3ε, a direct functional target of GR.^{35, 36}

Based on these observations, and without wishing to be bound by theory, a model in which severe trauma increases the amount of GR-FKBP51 binding, accompanied by lower GR S₂₁₁ phosphorylation, may be proposed. Both of these reduce GR-FKBP52 complex levels and reduce GR nuclear translocation, resulting in decreased GR transcriptional activity. These studies did not, however, conclude the exact mechanisms driving the increase in GR-FKBP51 complex levels. Without wishing to be bound by theory, these mechanisms are likely complex and multifaceted, and may include pathological trauma-induced abnormalities in the timing, extent and regulation of cortisol release, as well as genetic and epigenetic factors such as those previously described by Klengel et al.¹²

Typically, a stressful situation may be expected to result in cortisol release, which may increase GR S₂₁₁ phosphorylation^{33, 37} and may increase the transcriptional activity of GR. However, it is speculated here that genetic variants of GR and/or FKBP51 that increase affinity for each other, may also make the variation carriers more prone to PTSD by interfering with the normal GR S₂₁₁ phosphorylation response to cortisol release in times of stress. Alternatively, it is also possible that cortisol release patterns after trauma may be altered by various psychological factors such as childhood trauma,^{12, 38} perceived lack of control and guilt regarding actions during the traumatic event³⁹ or the degree and type of combat actions,⁴⁰ all potentially resulting in differential epigenetic regulation of cortisol-responsive genes. Abnormal cortisol signaling may then lead to excessive GR-FKBP51 complex formation. The literature provides conflicting evidence for the relationship between cortisol levels and dynamics in PTSD.⁴¹ Some data suggest that hypersensitivity to cortisol signaling can increase risk for PTSD.⁴² But there is also evidence that insufficient cortisol release can contribute to PTSD,^{38, 43} and that exogenous hydrocortisone treatment soon after a traumatic event may reduce the risk of developing PTSD.^{44, 45} This latter approach may be consistent with the effect of the peptide used in these studies in promoting GR functional effects. Given the range of cortisol responses to stress, the peptide, or other such agents, may represent a more reliable and/or predictable way to reduce PTSD risk after trauma than giving hydrocortisone, since it may act more directly on the GR-FKBP51 regulatory complex.

It is notable that the level of GR S₂₁₁ phosphorylation was comparable to that of the phosphomimetic mutant S_{211E}, and both have lower affinity for FKBP51 than the non-phosphorylated mutant S_{211A} GR. Without wishing to be bound by theory, these data suggest that the native state of GR S₂₁₁ may be constitutive phosphorylation, and that under pathological conditions, decreased GR S₂₁₁ phosphorylation may lead to increased GR-FKBP51 complex formation as part of the pathophysiology of PTSD. The reverse scenario may also be possible, that increased GR-FKBP51 complex formation may inhibit GR S₂₁₁ phosphorylation. Perhaps both of these two pathways may occur in parallel, and/or may have a synergistic, co-operative, or additive effect.

Efforts have been made to address some of the common limitations of this type of study, including the use of peripheral blood as the human tissue for studying a brain disorder, and the cross-species translation to mouse tissue samples. Peripheral blood was used to discover a protein complex biomarker for PTSD that was then applied to disrupt fear conditioning memory formation and recall in the mouse. These combined data provide strong evidence that the elevated protein complex detected in human blood from PTSD patients may indeed reflect an abnormality that when corrected in mouse brain, may disrupt fear memory paradigms relevant to PTSD in humans. The peripheral blood measurement of GR-FKBP51 levels was also validated as a proxy for brain by directly comparing protein samples from blood and brain in pooled mouse tissue. We found comparable relative changes in GR-FKBP51 levels for both tissue sources in the mouse, suggesting that the human blood samples did indeed provide a window into brain levels of this complex (FIG. 4G-H).

These results may be significant for several reasons. (1) Evidence for a direct protein interaction between the GR and FKBP51 proteins is provided that may represent a novel molecular mechanism for modulating GR function under stress, in addition to the previously-reported genetic interaction. (2) The binding sites for the GR-FKBP51 interaction have been determined, and an interfering peptide has been developed that may be useful not only as an experimental tool, but may also represent a potential treatment for PTSD. (3) Because the developed interfering peptide blocked the consolidation of fear memories, it is proposed that it may be used in an antidote paradigm, where patients exposed to severe trauma may be given the peptide as a prophylaxis against the potential future emergence of PTSD. In medicine, prevention is preferable to treatment, and these results suggest that the GR-FKBP51 complex may offer treatment options for both.

It has been theorized that the FKBP51 protein may somehow contribute to sequestering GR in the cytoplasm,^{16, 17, 18} but results herein provide what is believed to be the first direct evidence of such an interaction and formation of such a complex. The present inventors theorized that the GR-FKBP51 protein complex levels should be higher in patients with PTSD compared to unaffected control subjects, regardless of their FKBP51 genotype, and this is supported by the obtained data. A peptide-based inhibiting agent was synthesized that blocked the FKBP51-GR interaction, and substantially attenuated the behavioural response in mice exposed to strong fear-inducing stimuli (an animal model for PTSD). These experiments demonstrate what is believed to be a novel mechanism contributing to PTSD, and identify new treatment and/or diagnostic options for PTSD. Results indicate that agents which interfere with GR-FKBP51 complex may, for example, be administered to victims of traumatic events who show elevated levels of the GR-FKBP51 complex, and may prevent or reduce the later emergence of PTSD.

Methods

Human Subject Recruitment and Clinical Assessment

All human subject procedures were conducted in accordance with the protocol approved by the research ethics board at the Centre for Addiction and Mental Health (CAMH) in Toronto, Canada. PTSD patients (n=22) were recruited from an inpatient ward at CAMH and healthy controls (n=22) were separately recruited. Traumatic memory-related symptoms were assessed with the civilian version of the PTSD checklist (PCL-C), which is a self-report questionnaire that permits the scoring of both severity of post-traumatic symptoms, and the diagnosis of PTSD based on DSM-IV criteria.⁴⁶ PTSD subjects underwent a structured evaluation of their diagnosis using the Mini International Neuropsychiatric Interview (MINI).⁴⁷ Exclusion criteria were acute or chronic physical illnesses or systemic treatment with corticosteroids. Control subjects were excluded if they had a psychiatric diagnosis (including substance abuse) or had any serious medical illness (requiring ongoing medical treatment). Two 10 ml tubes (BD vacutainer, K2 EDTA) of peripheral blood were collected by venipuncture between 9 am to 11 am, immediately after the interview. Lymphocytes were isolated using Ficoll-Paque PLUS (GE Healthcare Life Sciences) and stored at −80° C. until further processing.

Mice

Male C57/BL6 mice, aged 7-week, were purchased from Charles Rivers Laboratories (Canada), and were given one week to acclimatize to the vivarium.

Some of the animals were later subjected to implantation of cannula into either amygdala (AP−1.5, LM−3.0, DV−4.8) or motor cortex (AP+2.1, LM−2.0, DV−1.0). Following surgeries, animals were allowed one week to recover before subjected to any experimental procedures.

HEK293T Cell Culture and DNA Transfection

HEK293T cells were maintained as a monolayer culture at 37° C. in Dulbecco's Modified Eagle Medium (DMEM) (Invitrogen) supplemented with 10% fetal bovine serum (Gibco). Cells were grown to 90% confluence before being transiently transfected with DNA constructs by X-treme GENE 9 transfection reagent (Roche), following the manufacturer's instructions. About 24-48 hrs after transfection, cells were used for experiments.

Primary Neuronal Culture

Cortical tissues from embryonic day (E14) mouse brains were dissected out, incubated with 0.25% trypsin for 15 min at 37° C., and dissociated by mechanical trituration. Neurons were then plated at a desired density onto glass coverslips previously coated with 0.1 mg/ml poly-d-lysine, and grown in Neurobasal medium with 1× Glutamax, 1×B27, 100 U/ml penicillin and 100 μg/ml streptomycin in an incubator (37° C., 5% CO₂). Either 10 μM of TAT-GRpep, 10 μM TAT-control peptide or saline were added in culture plates for 45 min in advance at 8 days in vitro (8 DIV).

Co-Immunoprecipitation and Western Blot

Co-immunoprecipitation and Western blot analyses were performed as previously described.^{24, 25, 26} For co-immunoprecipitation, 500˜1000 μg solubilized protein was extracted from whole mouse brain and incubated with primary antibody or control IgG (2˜4 μg) for 4 hrs at 4° C., followed by the addition of 25 μl protein A/G plus agarose (Santa Cruz Biotechnology) for 12 hrs. Pellets were washed, boiled for 5 min in SDS sample buffer and subjected to SDS-PAGE. 50˜100 μg of total protein extract was used as control in each experiment. After transfer of proteins into nitrocellulose, membranes were Western blotted with the primary antibodies. The intensity of protein level was quantified by densitometry (software: ImageLab, Bio-Rad).

The antibodies used were against GR (Santa Cruz Biotechnology, rabbit), FKBP51 (Santa Cruz Biotechnology, rabbit), GFP (Santa Cruz Biotechnology, rabbit) for immunoprecipitation and FKBP52 (Santa Cruz Biotechnology, mouse), Flag (Sigma-Aldrich, mouse), and GFP (Santa Cruz Biotechnology, mouse) for Western blot. The other antibodies were 14-3-3 ε (Santa Cruz Biotechnology, mouse), β-Actin (Cell Signaling, rabbit), anti-Histone H3 (Abcam, rabbit), and anti-phospho-GR (Ser211) (Cell signaling technology, rabbit).

GST Fusion Protein Constructs and TAT-GRpep Peptide

GST-fusion proteins encoding truncated GR fragments were amplified by PCR from full-length human cDNA clones. All constructs were sequenced to confirm the absence of PCR-generated errors. GST-fusion proteins were prepared from bacterial lysates with glutathione sepharose 4B beads as instructed by the manufacturer (Amersham).²⁵ To construct GST-fusion proteins encoding truncated GR, cDNA fragments were amplified by using PCR with specific primers. 5′ and 3′ oligonucleotides incorporated with specific restriction enzyme sites were used to facilitate sub-cloning into the pGEX-4T3 vector. To confirm appropriate splice fusion and correct nucleotide sequences, all constructs were re-sequenced.

TAT-GRpep peptide (AA sequence: GRKKRRQRRRPQSPWRSDLLIDENCLL) (SEQ ID NO: 9, see FIG. 10) was synthetically prepared.

Cued Fear Conditioning

Animals were first given a five-minute habituation to the apparatus (Habitest, Colbourne Instruments). Then, animals were exposed to 30 seconds of cue exposure (an in-house white light), which co-terminated with one second of foot-shock (0.5 mA). The cue exposure and foot-shock were repeated five times, intermitted by 5-minute inter-trial intervals. Following the conditioning, animals were subjected to 2-5 days of extinction before the assessment of their cued fear memory, which consisted of 15 rounds of 30-second cue exposure, with 30-second inter-trial intervals (adapted from Andero et al.).⁴⁸

The expression of fear (in a different context from training) was assessed 24 hours after the last extinction training. The assessment consisted of a three-minute period of habituation (absence of CS) and a sequent three-minute trial of conditioned cues. The duration of freezing behaviors was recorded by a blinded experimenter. Peptide treatment was either administered 2 hours after the conditioning or 30 minutes before the behavioral assessment.

Acute Brain Slices

Acute brain slices (350 μm thick) were prepared from C57Bl/6 mice with a McIlwain tissue chopper (Mickle Laboratory Engineering, Gomshall, United Kingdom). After being dissected from the brain, mouse cerebral hemispheres were left for 5 min in ice-cold artificial cerebrospinal fluid (aCSF) containing 126 mM NaCl, 2.5 mM KCl, 1 mM MgCl₂, 1 mM CaCl₂, 1.25 mM KH₂PO₄, 26 mM NaHCO₃ and 20 mM glucose, which was bubbled continuously with carbogen (95% O₂/5% CO₂). Freshly cut slices were placed in an incubating chamber with carbogenated aCSF and recovered from stress at 37° C. for 1 hour. Slices were then treated with TAT or TAT-GRpep and harvested for Western blot analysis.

Plasmid Mutation

Mutants of Flag-GR and GST-GR-NT were created with the QuickChange site-directed mutagenesis kit (Stratagene, La Jolla, Calif.). All mutants were confirmed by DNA sequencing.

Surface Plasmon Resonance

The binding of FKBP51 TPR domain to GR and its phosphorylation mutants was determined by surface plasmon resonance using a Reichert SR7500DC instrument. Experiments were conducted at 25° C. with 2000 RU HEK293 expressed FLAG-tagged GR bound onto a FLAG antibody-coated Dextran 50,000 Da chip. Running buffer (PBS, pH 7.4 with 0.005% Tween 20) was used with a flow rate of 25 μl/min. With association time of 300s and dissociation of 240s, binding signals were determined by injecting various concentrations of the GST-FKBP51_TPR.

Nuclear Protein Isolation Assay

Nuclear protein isolation assays were performed in acute mouse brain slices or mouse brain tissue using the Nuclear Protein Extraction Kit (Bio Basic Inc., Amherst, N.Y.). Briefly, after treatment with TAT or TAT-GRpep, mouse brain slices were harvested for nuclear protein extraction according to the manufacturer's instructions.

Nuclear protein isolation assays were performed in human lymphocytes using the NE-PER Nuclear and Cytoplasmic Extraction Reagents Kit (Thermo Scientific).

Statistics

Densitometry of protein levels were normalized after being divided by the mean value of control levels from the same blot. Results for each sample were presented as the percentage of control group. The independent samples t-test, one-way ANOVA test followed by Dunnett post hoc test and two-way ANOVA were used to compare different experimental groups. Data was shown as mean S.E.M.

Immunocytochemistry

Cultured neurons were fixed in 4% PFA/4% sucrose, permeabilized with 0.1M PBS containing 0.1% Triton X-100 for 10 min, and blocked for 1 hour with 1% bovine serum albumin in PBS at room temperature. They were incubated with primary antibodies overnight at 4° C. and secondary antibodies for 1 hour at room temperature. The following primary antibodies were used: anti-GR (1:200; Thermo), anti-FKBP51 (1:200; Abcam) and anti-FKBP52 (1:200; Abcam). Fluorescent secondary antibodies conjugated to Alexa 488 or 594 (1:200; Life technologies) were used for detection of primary antibodies. DAPI was used to stain cell nuclei.

One or more illustrative embodiments have been described by way of example. It will be understood to persons skilled in the art that a number of variations and modifications can be made without departing from the scope of the invention as defined in the claims.

### REFERENCES

1. American Psychiatric Association. Diagnostic and statistical manual of mental disorders 5th edn. American Psychiatric Publishing (2013).
2. Lawson N R. Posttraumatic stress disorder in combat veterans. JAAPA 27, 18-22 (2014).
3. Johansen V A, Eilertsen D E, Nordanger D, Weisaeth L. Prevalence, comorbidity and stability of post-traumatic stress disorder and anxiety and depression symptoms after exposure to physical assault: an 8-year prospective longitudinal study. Nord J Psychiatry 67, 69-80 (2013).
4. Smith T C, Wingard D L, Ryan M A, Kritz-Silverstein D, Slymen D J, Sallis J F. PTSD prevalence, associated exposures, and functional health outcomes in a large, population-based military cohort. Public Health Rep 124, 90-102 (2009).
5. Van Ameringen M, Mancini C, Patterson B, Boyle M H. Post-traumatic stress disorder in Canada. CNS Neurosci Ther 14, 171-181 (2008).
6. Jonas D E, et al. Psychological and Pharmacological Treatments for Adults With Posttraumatic Stress Disorder (PTSD). AHRQ Comparative Effectiveness Reviews, (2013).
7. Bisson J I, Roberts N P, Andrew M, Cooper R, Lewis C. Psychological therapies for chronic post-traumatic stress disorder (PTSD) in adults. Cochrane Database Syst Rev 12, CD003388 (2013).
8. Hetrick S E, Purcell R, Garner B, Parslow R. Combined pharmacotherapy and psychological therapies for post traumatic stress disorder (PTSD). Cochrane Database Syst Rev, CD007316 (2010).
9. Murdoch M, et al. Long-term outcomes of disability benefits in US veterans with posttraumatic stress disorder. Arch Gen Psychiatry 68, 1072-1080 (2011).
10. Zatzick D, et al. A national US study of posttraumatic stress disorder, depression, and work and functional outcomes after hospitalization for traumatic injury. Ann Surg 248, 429-437 (2008).
11. Shalev A Y. Posttraumatic stress disorder and stress-related disorders. Psychiatr Clin North Am 32, 687-704 (2009).
12. Klengel T, et al. Allele-specific FKBP5 DNA demethylation mediates gene-childhood trauma interactions. Nat Neurosci 16, 33-41 (2013).
13. Binder E B, et al. Association of FKBP5 polymorphisms and childhood abuse with risk of posttraumatic stress disorder symptoms in adults. JAMA 299, 1291-1305 (2008).
14. Mehta D, et al. Using polymorphisms in FKBP5 to define biologically distinct subtypes of posttraumatic stress disorder: evidence from endocrine and gene expression studies. Arch Gen Psychiatry 68, 901-910 (2011).
15. Szyf M. How do environments talk to genes? Nat Neurosci 16, 2-4 (2013).
16. Gallo L I, Lagadari M, Piwien-Pilipuk G, Galigniana M D. The 90-kDa heat-shock protein (Hsp90)-binding immunophilin FKBP51 is a mitochondrial protein that translocates to the nucleus to protect cells against oxidative stress. J Biol Chem 286, 30152-30160 (2011).
17. Zhang X, Clark A F, Yorio T. FK506-binding protein 51 regulates nuclear transport of the glucocorticoid receptor beta and glucocorticoid responsiveness. Invest Ophthalmol Vis Sci 49, 1037-1047 (2008).
18. Wochnik G M, Ruegg J, Abel G A, Schmidt U, Holsboer F, Rein T. FK506-binding proteins 51 and 52 differentially regulate dynein interaction and nuclear translocation of the glucocorticoid receptor in mammalian cells. J Biol Chem 280, 4609-4616 (2005).
19. Golub Y, Mauch C P, Dahlhoff M, Wotjak C T. Consequences of extinction training on associative and non-associative fear in a mouse model of Posttraumatic Stress Disorder (PTSD). Behav Brain Res 205, 544-549 (2009).
20. Brinks V, de Kloet E R, Oitzl M S. Strain specific fear behaviour and glucocorticoid response to aversive events: modelling PTSD in mice. Prog Brain Res 167, 257-261 (2008).
21. Siegmund A, Wotjak C T. A mouse model of posttraumatic stress disorder that distinguishes between conditioned and sensitised fear. J Psychiatr Res 41, 848-860 (2007).
22. Ressler K J, et al. Post-traumatic stress disorder is associated with PACAP and the PAC1 receptor. Nature 470, 492-497 (2011).
23. Gillespie C F, et al. Trauma exposure and stress-related disorders in inner city primary care patients. Gen Hosp Psychiatry 31, 505-514 (2009).
24. Su P, et al. A dopamine D2 receptor-DISC1 protein complex may contribute to antipsychotic-like effects. Neuron 84, 1302-1316 (2014).
25. Lee F J, Pei L, Moszczynska A, Vukusic B, Fletcher P J, Liu F. Dopamine transporter cell surface localization facilitated by a direct interaction with the dopamine D2 receptor. EMBO J 26, 2127-2136 (2007).
26. Pei L, et al. Uncoupling the dopamine D1-D2 receptor complex exerts antidepressant-like effects. Nat Med 16, 1393-1395 (2010).
27. Mahan A L, Mou L, Shah N, Hu J H, Worley P F, Ressler K J. Epigenetic modulation of Homer1a transcription regulation in amygdala and hippocampus with pavlovian fear conditioning. J Neurosci 32, 4651-4659 (2012).
28. Anbalagan M, Huderson B, Murphy L, Rowan B G. Post-translational modifications of nuclear receptors and human disease. Nuclear receptor signaling 10, e001 (2012).
29. Oakley R H, Cidlowski J A. The biology of the glucocorticoid receptor: new signaling mechanisms in health and disease. J Allergy Clin Immunol 132, 1033-1044 (2013).
30. Wallace A D, Cidlowski J A. Proteasome-mediated glucocorticoid receptor degradation restricts transcriptional signaling by glucocorticoids. The Journal of biological chemistry 276, 42714-42721 (2001).
31. Webster J C, Jewell C M, Bodwell J E, Munck A, Sar M, Cidlowski J A. Mouse glucocorticoid receptor phosphorylation status influences multiple functions of the receptor protein. The Journal of biological chemistry 272, 9287-9293 (1997).
32. Miller A L, et al. p38 Mitogen-activated protein kinase (MAPK) is a key mediator in glucocorticoid-induced apoptosis of lymphoid cells: correlation between p38 MAPK activation and site-specific phosphorylation of the human glucocorticoid receptor at serine 211. Mol Endocrinol 19, 1569-1583 (2005).
33. Wang Z, Frederick J, Garabedian M J. Deciphering the phosphorylation “code” of the glucocorticoid receptor in vivo. J Biol Chem 277, 26573-26580 (2002).
34. Jonas D E, et al. In: Psychological and Pharmacological Treatments for Adults With Posttraumatic Stress Disorder (PTSD) (ed\hat{ }(eds) (2013).
35. Ayyar V S, Almon R R, DuBois D C, Sukumaran S, Qu J, Jusko W J. Functional proteomic analysis of corticosteroid pharmacodynamics in rat liver: Relationship to hepatic stress, signaling, energy regulation, and drug metabolism. J Proteomics 160, 84-105 (2017).
36. Phuc Le P, et al. Glucocorticoid receptor-dependent gene regulatory networks. PLoS Genet 1, e16 (2005).
37. Galliher-Beckley A J, Cidlowski J A. Emerging roles of glucocorticoid receptor phosphorylation in modulating glucocorticoid hormone action in health and disease. IUBMB Life 61, 979-986 (2009).
38. Steudte-Schmiedgen S, et al. Hair cortisol concentrations and cortisol stress reactivity predict PTSD symptom increase after trauma exposure during military deployment. Psychoneuroendocrinology 59, 123-133 (2015).
39. Norman S B, et al. Trauma related guilt cognitions partially mediate the relationship between PTSD symptom severity and functioning among returning combat veterans. J Psychiatr Res 100, 56-62 (2018).
40. Van Winkle E P, Safer M A. Killing versus witnessing in combat trauma and reports of PTSD symptoms and domestic violence. J Trauma Stress 24, 107-110 (2011).
41. Galatzer-Levy I R, Ma S, Statnikov A, Yehuda R, Shalev A Y. Utilization of machine learning for prediction of post-traumatic stress: a re-examination of cortisol in the prediction and pathways to non-remitting PTSD. Transl Psychiatry 7, e0 (2017).
42. Hauer D, Kaufmann I, Strewe C, Briegel I, Campolongo P, Schelling G. The role of glucocorticoids, catecholamines and endocannabinoids in the development of traumatic memories and posttraumatic stress symptoms in survivors of critical illness. Neurobiol Learn Mem 112, 68-74 (2014).
43. Schelling G. Effects of stress hormones on traumatic memory formation and the development of posttraumatic stress disorder in critically ill patients. Neurobiol Learn Mem 78, 596-609 (2002).
44. Schelling G, Roozendaal B, De Quervain D J. Can posttraumatic stress disorder be prevented with glucocorticoids? Ann N Y Acad Sci 1032, 158-166 (2004).
45. Birur B, Math S B, Fargason R E. A Review of Psychopharmacological Interventions Post-Disaster to Prevent Psychiatric Sequelae. Psychopharmacol Bull 47, 8-26 (2017).
46. Ruggiero K J, Del Ben K, Scotti J R, Rabalais A E. Psychometric properties of the PTSD Checklist-Civilian Version. J Trauma Stress 16, 495-502 (2003).
47. Sheehan D V, et al. The Mini-International Neuropsychiatric Interview (M.I.N.I.): the development and validation of a structured diagnostic psychiatric interview for DSM-IV and ICD-10. J Clin Psychiatry 59 Suppl 20, 22-33; quiz 34-57 (1998).
48. Andero R, Heldt S A, Ye K, Liu X, Armario A, Ressler K J. Effect of 7,8-dihydroxyflavone, a small-molecule TrkB agonist, on emotional learning. Am J Psychiatry 168, 163-172 (2011).

All references cited in this section, and elsewhere throughout the specification, are herein incorporated by reference in their entireties.

1. A method for preventing or treating post-traumatic stress disorder (PTSD) in a subject in need thereof, said method comprising:
administering an agent which inhibits formation of a glucocorticoid receptor (GR)-FK506 Binding Protein 51 (FKBP51) complex (GR-FKBP51 complex), or which disrupts already formed GR-FKBP51 complex, to the subject;
thereby reducing a level of GR-FKBP51 complex in the subject and preventing or treating the PTSD.

2. The method of claim 1, wherein the agent comprises:
a competitive binder for a region of GR which binds FKBP51 in the GR-FKBP51 complex; or
a competitive binder for a region of FKBP51 which binds GR in the GR-FKBP51 complex.

3. The method of claim 1, wherein the agent comprises a mimic of an N-terminal region of GR (GR_{NT}; SEQ ID NO: 2) which interacts with FKBP51 in the GR-FKBP51 complex.

4. The method of claim 3, wherein the agent comprises a mimic of a GR_{NT-4} (SEQ ID NO: 3) or GR_{NT-4-1} (SEQ ID NO: 4) region of GR.

5. The method of claim 1, wherein the agent comprises a polypeptide having at least 80% sequence identity to the amino acid sequence S₂₁₁-L₂₂₅ of GR (SEQ ID NO: 4).

6. (canceled)

7. (canceled)

8. The method of claim 1, wherein the agent comprises a mimic of a tetratricopeptide repeat (TPR) domain (FKBP51_{TPR}; SEQ ID NO: 6) of FKBP1 which interacts with GR in the GR-FKBP51 complex.

9. The method of claim 8, wherein the agent comprises a mimic of a FKBP51_{TPR3} region of FKBP51 (SEQ ID NO: 7).

10. The method of claim 1, wherein the agent comprises a polypeptide having at least 80% sequence identity to the amino acid sequence of the FKBP51_{TPR3} region (SEQ ID NO: 7).

11.-15. (canceled)

16. The method of claim 1, wherein the agent comprises a polypeptide having at least 80% sequence identity to the amino acid sequence GRKKRRQRRRPQSPWRSDLLIDENCLL (SEQ ID NO: 9).

17. The method of claim 1, wherein the agent comprises a polypeptide having the amino acid sequence GRKKRRQRRRPQSPWRSDLLIDENCLL (SEQ ID NO: 9).

18. The method of claim 1, wherein the method further comprises the following step, performed before the step of administering:
measuring a level of GR-FKBP51 complex in the subject, comparing the measured level to a reference or control level representative of a non-PTSD condition, and identifying the subject as a candidate for treatment where the measured level of GR-FKBP51 complex in the subject is elevated relative to the reference or control level.

19.-46. (canceled)

47. A method for diagnosing a subject as having, or being at risk of developing, a post-traumatic stress disorder (PTSD), said method comprising:
measuring a level of a glucocorticoid receptor (GR)-FK506 Binding Protein 51 (FKBP51) complex (GR-FKBP51 complex) in the subject;
comparing the measured level to a reference or control level representative of a non-PTSD condition; and
identifying the subject as having, or being at risk of developing, PTSD where the measured level of GR-FKBP51 complex in the subject is elevated relative to the reference or control level.

48. (canceled)

49. (canceled)

50. The method of claim 47, wherein the step of measuring comprises ELISA, co-immunoprecipitation, and/or Western blotting to quantify the level of GR-FKBP51 complex.

51.-68. (canceled)

69. A polypeptide comprising a polypeptide sequence having at least 80% sequence identity to the amino acid sequence S₂₁₁-L₂₂₅ of GR (SEQ ID NO: 4).

70. The polypeptide of claim 69, wherein an amino acid residue of the polypeptide which corresponds to position S₂₁₁ of GR comprises a wild-type (WT) residue, an S_{211A} mutant residue, or an S_{211E} mutant residue.

71. The polypeptide of claim 69, wherein the polypeptide comprises a polypeptide sequence having the amino acid sequence S₂₁₁-L₂₂₅ of GR (SEQ ID NO: 4), or at least any 7 consecutive amino acids thereof.

72. (canceled)

73. (canceled)

74. (canceled)

75. (canceled)

76. The polypeptide of claim 69, wherein the polypeptide comprises a polypeptide sequence having at least 80% sequence identity to the amino acid sequence GRKKRRQRRRPQSPWRSDLLIDENCLL (SEQ ID NO: 9).

77. The polypeptide of claim 69, wherein the polypeptide comprises a polypeptide sequence having the amino acid sequence GRKKRRQRRRPQSPWRSDLLIDENCLL (SEQ ID NO: 9).

78.-83. (canceled)

84. A pharmaceutical composition comprising the polypeptide of claim 69 and a pharmaceutically acceptable excipient, diluent, or carrier.

85.-95. (canceled)


SEQUENCE LISTING (26 sequences)

SEQ ID NO: 1 - protein, 777 aa, Homo sapiens
MDSKESLTPGREENPSSVLAQERGDVMDFYKTLRGGATVKVSASSPSLAVASQSDSKQRRLLVDFPKGSVSNAQQPDLSKAVSLSMGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 2 - protein, 420 aa, Artificial Sequence
  Amino Acid Sequence of N-terminal Region of Human Glucocorticoid Receptor (GR)
MDSKESLTPGREENPSSVLAQERGDVMDFYKTLRGGATVKVSASSPSLAVASQSDSKQRRLLVDFPKGSVSNAQQPDLSKAVSLSMGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNESPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKL

SEQ ID NO: 3 - protein, 70 aa, Artificial Sequence
  Amino Acid Sequence of GRNT-4 Region of Human Glucocorticoid Receptor (GR)
SPWRSDLLIDENCLLSPLAGEDDSFLLEGNSNEDCKPLILPDTKPKIKDNGDLVLSSPSNVTLPQVKTEK

SEQ ID NO: 4 - protein, 15 aa, Artificial Sequence
  Amino Acid Sequence of S211-L225 Region (GRNT-4-1 Region) of Human Glucocorticoid Receptor (GR)
SPWRSDLLIDENCLL

SEQ ID NO: 5 - protein, 457 aa, Homo Sapiens
MTTDEGAKNNEESPTATVAEQGEDITSKKDRGVLKIVKRVGNGEETPMIGDKVYVHYKGKLSNGKKFDSSHDRNEPFVFSLGKGQVIKAWDIGVATMKKGEICHLLCKPEYAYGSAGSLPKIPSNATLFFEIELLDFKGEDLFEDGGIIRRTKRKGEGYSNPNEGATVEIHLEGRCGGRMFDCRDVAFTVGEGEDHDIPIGIDKALEKMQREEQCILYLGPRYGFGEAGKPKFGIEPNAELIYEVTLKSFEKAKESWEMDTKEKLEQAAIVKEKGTVYFKGGKYMQAVIQYGKIVSWLEMEYGLSEKESKASESFLLAAFLNLAMCYLKLREYTKAVECCDKALGLDSANEKGLYRRGEAQLLMNEFESAKGDFEKVLEVNPQNKAARLQISMCQKKAKEHNERDRRIYANMFKKFAEQDAKEEANKAMGKKTSEGVTNEKGTDSQAMEEEKPEGHV

SEQ ID NO: 6 - protein, 200 aa, Artificial Sequence
  Amino Acid Sequence of the FKBP51TPR Domain of Human FK506 Binding Protein 51 (FKBP51)
EMDTKEKLEQAAIVKEKGTVYFKGGKYMQAVIQYGKIVSWLEMEYGLSEKESKASESFLLAAFLNLAMCYLKLREYTKAVECCDKALGLDSANEKGLYRRGEAQLLMNEFESAKGDFEKVLEVNPQNKAARLQISMCQKKAKEHNERDRRIYANMFKKFAEQDAKEEANKAMGKKTSEGVTNEKGTDSQAMEEEKPEGHV

SEQ ID NO: 7 - protein, 50 aa, Artificial Sequence
  Amino Acid Sequence of the FKBP51TPR3 Domain of Human FK506 Binding Protein 51 (FKBP51)
GEAQLLMNEFESAKGDFEKVLEVNPQNKAARLQISMCQKKAKEHNERDRR

SEQ ID NO: 8 - protein, 12 aa, Artificial Sequence
  Amino Acid Sequence of the Cell-Membrane Transduction Domain of HIV-Type 1 Transactivator of Transcription (TAT) Protein
GRKKRRQRRRPQ

SEQ ID NO: 9 - protein, 27 aa, Artificial Sequence
  Amino Acid Sequence of TAT-GRpep Peptide
GRKKRRQRRRPQSPWRSDLLIDENCLL

SEQ ID NO: 10 - protein, 777 aa, Artificial Sequence
  Amino Acid Sequence of S211A GR Mutant (non-phosphorylated GR mutant)
MDSKESLTPGREENPSSVLAQERGDVMDFYKTLRGGATVKVSASSPSLAVASQSDSKQRRLLVDFPKGSVSNAQQPDLSKAVSLSMGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNEAPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 11 - protein, 777 aa, Artificial Sequence
  Amino Acid Sequence of S211E GR Mutant (phosphomimetic GR mutant)
MDSKESLTPGREENPSSVLAQERGDVMDFYKTLRGGATVKVSASSPSLAVASQSDSKQRRLLVDFPKGSVSNAQQPDLSKAVSLSMGLYMGETETKVMGNDLGFPQQGQISLSSGETDLKLLEESIANLNRSTSVPENPKSSASTAVSAAPTEKEFPKTHSDVSSEQQHLKGQTGTNGGNVKLYTTDQSTFDILQDLEFSSGSPGKETNEEPWRSDLLIDENCLLSPLAGEDDSFLLEGNSNEDCKPLILPDTKPKIKDNGDLVLSSPSNVTLPQVKTEKEDFIELCTPGVIKQEKLGTVYCQASFPGANIIGNKMSAISVHGVSTSGGQMYHYDMNTASLSQQQDQKPIFNVIPPIPVGSENWNRCQGSGDDNLTSLGTLNFPGRTVFSNGYSSPSMRPDVSSPPSSSSTATTGPPPKLCLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 12 - protein, 357 aa, Artificial Sequence
  Amino Acid Sequence of GRCT Fragment
CLVCSDEASGCHYGVLTCGSCKVFFKRAVEGQHNYLCAGRNDCIIDKIRRKNCPACRYRKCLQAGMNLEARKTKKKIKGIQQATTGVSQETSENPGNKTIVPATLPQLTPTLVSLLEVIEPEVLYAGYDSSVPDSTWRIMTTLNMLGGRQVIAAVKWAKAIPGFRNLHLDDQMTLLQYSWMFLMAFALGWRSYRQSSANLLCFAPDLIINEQRMTLPCMYDQCKHMLYVSSELHRLQVSYEEYLCMKTLLLLSSVPKDGLKSQELFDEIRMTYIKELGKAIVKREGNSSQNWQRFYQLTKLLDSMHEVVENLLNYCFQTFLDKTMSIEFPEMLAEIITNQIPKYSNGNIKKLLFHQK

SEQ ID NO: 13 - protein, 70 aa, Artificial Sequence
  Amino Acid Sequence of GRNT-1 Fragment
MDSKESLTPGREENPSSVLAQERGDVMDFYKTLRGGATVKVSASSPSLAVASQSDSKQRRLLVDFPKGSV

SEQ ID NO: 14 - protein, 70 aa, Artificial Sequence
  Amino Acid Sequence of GRNT-2 Fragment
SNAQQPDLSKAVSLSMGLYMGETETKVMGNDLGFPQQGQISLSSGETDLKLLEESIANLNRSTSVPENPK

SEQ ID NO: 15 - protein, 70 aa, Artificial Sequence
  Amino Acid Sequence of GRNT-2 Fragment
SSASTAVSAAPTEKEFPKTHSDVSSEQQHLKGQTGTNGGNVKLYTTDQSTFDILQDLEFSSGSPGKETNE

SEQ ID NO: 16 - protein, 70 aa, Artificial Sequence
  Amino Acid Sequence of GRNT-5 Fragment
EDFIELCTPGVIKQEKLGTVYCQASFPGANIIGNKMSAISVHGVSTSGGQMYHYDMNTASLSQQQDQKPI

SEQ ID NO: 17 - protein, 70 aa, Artificial Sequence
  Amino Acid Sequence of GRNT-6 Fragment
FNVIPPIPVGSENWNRCQGSGDDNLTSLGTLNFPGRTVFSNGYSSPSMRPDVSSPPSSSSTATTGPPPKL

SEQ ID NO: 18 - protein, 15 aa, Artificial Sequence
  Amino Acid Sequence of GRNT-4-2 Fragment
LLSPLAGEDDSFLLE

SEQ ID NO: 19 - protein, 15 aa, Artificial Sequence
  Amino Acid Sequence of GRNT-4-3 Fragment
EGNSNEDCKPLILPD

SEQ ID NO: 20 - protein, 15 aa, Artificial Sequence
  Amino Acid Sequence of GRNT-4-4 Fragment
DTKPKIKDNGDLVLS

SEQ ID NO: 21 - protein, 15 aa, Artificial Sequence
  Amino Acid Sequence of GRNT-4-5 Fragment
SSPSNVTLPQVKTEK

SEQ ID NO: 22 - protein, 143 aa, Artificial Sequence
  Amino Acid Sequence of FKBP51FK1 Fragment
MTTDEGAKNNEESPTATVAEQGEDITSKKDRGVLKIVKRVGNGEETPMIGDKVYVHYKGKLSNGKKFDSSHDRNEPFVFSLGKGQVIKAWDIGVATMKKGEICHLLCKPEYAYGSAGSLPKIPSNATLFFEIELLDFKGEDLF

SEQ ID NO: 23 - protein, 114 aa, Artificial Sequence
  Amino Acid Sequence of FKBP51FK2 Fragment
EDGGIIRRTKRKGEGYSNPNEGATVEIHLEGRCGGRMFDCRDVAFTVGEGEDHDIPIGIDKALEKMQREEQCILYLGPRYGFGEAGKPKFGIEPNAELIYEVTLKSFEKAKESW

SEQ ID NO: 24 - protein, 50 aa, Artificial Sequence
  Amino Acid Sequence of FKBP51TPR-1 Fragment
EMDTKEKLEQAAIVKEKGTVYFKGGKYMQAVIQYGKIVSWLEMEYGLSEK

SEQ ID NO: 25 - protein, 50 aa, Artificial Sequence
  Amino Acid Sequence of FKBP51TPR-2 Fragment
ESKASESFLLAAFLNLAMCYLKLREYTKAVECCDKALGLDSANEKGLYRR

SEQ ID NO: 26 - protein, 50 aa, Artificial Sequence
  Amino Acid Sequence of FKBP51TPR-4 Fragment
IYANMFKKFAEQDAKEEANKAMGKKTSEGVTNEKGTDSQAMEEEKPEGHV